(19) United States
(12) Patent Application Publication
(10) Pub. No.: US 2024/0366110 A1
(43) Pub. Date: Nov. 7, 2024

(54) HUMAN BODY POSTURE RECOGNITION SYSTEM

(71) Applicant: SHENZHEN SHOKZ CO., LTD., Shenzhen (CN)

(72) Inventors: Wenjun DENG, Shenzhen (CN); Xin ZHOU, Shenzhen (CN); Lei SU, Shenzhen (CN); Meiqi LI, Shenzhen (CN)

(73) Assignee: SHENZHEN SHOKZ CO., LTD., Shenzhen (CN)

(21) Appl. No.: 18/770,635

(22) Filed: Jul. 11, 2024

Related U.S. Application Data
(63) Continuation of application No. PCT/CN2022/096684, filed on Jun. 1, 2022.

(51) Int. Cl. A61B 5/11; A61B 5/00

(52) U.S. Cl. CPC A61B 5/1116; A61B 5/1126; A61B 5/6804

(57) ABSTRACT

One or more embodiments of the present disclosure relate to a human body posture recognition system, comprising: at least one group of ultrasonic sensors, each group of ultrasonic sensors including an ultrasonic transmitter configured to transmit ultrasonic waves and an ultrasonic receiver configured to receive the ultrasonic waves. The ultrasonic transmitter and the ultrasonic receiver are located at different parts of the body of a user, respectively; and a processor configured to, on the basis of location information of the ultrasonic transmitter and the ultrasonic receiver, information of the ultrasonic waves transmitted by the ultrasonic transmitter, and information of the ultrasonic waves received by the ultrasonic receiver, recognize the posture of the user.

### CROSS-REFERENCE TO RELATED APPLICATIONS

This application is a continuation of International Patent Application No. PCT/CN2022/096684, filed on Jun. 1, 2022, the entire contents of which are hereby incorporated by reference.

### TECHNICAL FIELD

The present disclosure relates to the field of posture recognition technology, and in particular, to a human body posture recognition system.

### BACKGROUND

With the continuous development of science and technology, human posture recognition has been widely used in many fields such as sports and health, gaming, medical and healthcare, and wearable electronic devices. Currently, human posture recognition may require the use of a variety of sensors including infrared sensors, inertial sensors, and so on. However, the accuracy of human posture recognition using infrared sensors or inertial sensors is low, which results in a high difficulty in the application of human posture recognition.

Therefore, the present disclosure hopes to provide a human body posture recognition system to improve the accuracy of human posture recognition.

### SUMMARY

Embodiments of the present disclosure provide a human body posture recognition system, comprising at least one group of ultrasonic sensors, each group of ultrasonic sensors including an ultrasonic transmitter configured to transmit ultrasonic waves and an ultrasonic receiver configured to receive the ultrasonic waves. The ultrasonic transmitter and the ultrasonic receiver may be located at different parts of a user's body; and a processor configured to recognize a posture of the user based on location information of the ultrasonic transmitter and the ultrasonic receiver, information of the ultrasonic waves transmitted by the ultrasonic transmitter, and information of the ultrasonic waves received by the ultrasonic receiver.

In some embodiments, the system may further comprise a garment, and the at least one group of ultrasonic sensors is integrated into the garment.

In some embodiments, the ultrasonic transmitter and the ultrasonic receiver may be disposed at a sleeve or a trouser leg of the garment, and when the user wears the garment, the ultrasonic transmitter and the ultrasonic receiver may be located at two opposite limbs corresponding to an elbow joint or a knee joint of the user, respectively.

In some embodiments, the recognizing the posture of the user based on the location information of the ultrasonic transmitter and the ultrasonic receiver, the information of the ultrasonic waves transmitted by the ultrasonic transmitter, and the information of the ultrasonic waves received by the ultrasonic receiver may include obtaining a distance between the ultrasonic transmitter and the ultrasonic receiver based on the information of the ultrasonic waves transmitted by the ultrasonic transmitter and the information of the ultrasonic waves received by the ultrasonic receiver; and obtaining a bending angle between the two opposite limbs corresponding to the elbow joint or the knee joint based on the distance between the ultrasonic transmitter and the ultrasonic receiver, and the location information of the ultrasonic transmitter and the ultrasonic receiver.

In some embodiments, each group of ultrasonic sensors may include an ultrasonic transmitter and at least two ultrasonic receivers, the at least two ultrasonic receivers including a first ultrasonic receiver and a second ultrasonic receiver, and the recognizing the posture of the user based on the location information of the ultrasonic transmitter and the ultrasonic receiver, the information of the ultrasonic waves transmitted by the ultrasonic transmitter, and the information of the ultrasonic waves received by the ultrasonic receiver may include determining a first distance between the ultrasonic transmitter and the first ultrasonic receiver and a second distance between the ultrasonic transmitter and the second ultrasonic receiver based on the information of the ultrasonic waves transmitted by the ultrasonic transmitter, information of ultrasonic waves received by the first ultrasonic receiver, and information of ultrasonic waves received by the second ultrasonic receiver; and determining a rotation angle between the two opposite limbs corresponding to the elbow joint or the knee joint based on the first distance and the second distance.

In some embodiments, inner sides of the two opposite limbs corresponding to the elbow joint or the knee joint refer to two opposite lateral portions of the limb corresponding to the elbow joint or the knee joint when the elbow joint or the knee joint is in a bending state, and the ultrasonic transmitter and the ultrasonic receiver are located at the inner sides of the two opposite limbs corresponding to the elbow joint or the knee joint.

In some embodiments, the distance between the ultrasonic transmitter and the ultrasonic receiver may be no less than 10 cm when the user straightens his or her arm or leg.

In some embodiments, the ultrasonic transmitter may include an output end for transmitting the ultrasonic waves, and the output end is away from the garment; the ultrasonic receiver may include a receiving end for receiving the ultrasonic waves, and the receiving end is away from the garment; and an angle between a plane in which the output end is located and a plane in which the receiving end is located may be not greater than 170°.

In some embodiments, the ultrasonic transmitter may be disposed obliquely relative to a limb contact site on the user below the ultrasonic transmitter and disposed at an inclination toward the ultrasonic receiver, and an angle between a normal direction of the output end and a normal direction of the limb contact site on the user below the ultrasonic transmitter may be not less than 5°.

In some embodiments, the ultrasonic receiver may be disposed relative to the limb contact site on the user below the ultrasonic receiver and disposed at an inclination toward the ultrasonic transmitter, and an angle between a normal direction of the receiving end and a normal direction of the limb contact site on the user below the ultrasonic receiver may be not less than 5°.

In some embodiments, the ultrasonic transmitter may be located in the garment at a location corresponding to an upper arm of a human body, the ultrasonic receiver may be located in the garment at a location corresponding to a torso site of the human body, and the ultrasonic transmitter and the ultrasonic receiver may cooperate to recognize a posture of the upper arm relative to the torso site.

In some embodiments, the posture of the upper arm relative to the torso site may include an angle of the upper arm relative to the torso site, and the recognizing the posture of the upper arm relative to the torso site may include obtaining a distance between the ultrasonic transmitter and the ultrasonic receiver based on the information of the ultrasonic waves transmitted by the ultrasonic transmitter and the information of the ultrasonic waves received by the ultrasonic receiver, and determining the angle of the upper arm relative to the torso site based on the distance between the ultrasonic transmitter and the ultrasonic receiver and the location information of the ultrasonic transmitter and the ultrasonic receiver.

In some embodiments, the ultrasonic transmitter may include at least two ultrasonic transmitters and the ultrasonic receiver may include at least three ultrasonic receivers, and the recognizing the posture of the upper arm relative to the torso site may include determining location change information of the at least two ultrasonic transmitters based on location information of the at least three ultrasonic receivers, and recognizing a motion state of the upper arm relative to the torso site based on the location change information of the at least two ultrasonic transmitters.

In some embodiments, the at least three ultrasonic receivers may be not in a same straight line.

In some embodiments, the at least two ultrasonic transmitters may be spaced no less than 1 cm apart from each other, and the at least three ultrasonic receivers may be spaced no less than 1 cm apart from each other.

In some embodiments, in the garment, the location corresponding to the torso site of the human body may include at least one of a front side of a left shoulder, a front side of a right shoulder, a left lumbar side, a right lumbar side, or a chest.

In some embodiments, the ultrasonic transmitter and the ultrasonic receiver may be located at two trouser legs of the garment, respectively, and the recognizing the posture of the user based on the location information of the ultrasonic transmitter and the ultrasonic receiver, the information of the ultrasonic waves transmitted by the ultrasonic transmitter, and the information of the ultrasonic waves received by the ultrasonic receiver may include obtaining a distance between the ultrasonic transmitter and the ultrasonic receiver based on the information of the ultrasonic waves transmitted by the ultrasonic transmitter and the information of the ultrasonic waves received by the ultrasonic receiver; and determining a posture of a leg of the user based on the distance between the ultrasonic transmitter and the ultrasonic receiver, and the location information of the ultrasonic transmitter and the ultrasonic receiver.

In some embodiments, each group of ultrasonic sensors may include a plurality of ultrasonic transmitters, and the plurality of ultrasonic transmitters may transmit ultrasonic waves at time intervals.

In some embodiments, two adjacent ultrasonic transmitters may transmit ultrasonic waves at a time interval greater than 2.9 ms.

In some embodiments, each group of ultrasonic sensors may include a plurality of ultrasonic transmitters, and the plurality of ultrasonic transmitters may transmit ultrasonic waves at different frequencies.

In some embodiments, each group of ultrasonic sensors may include a plurality of ultrasonic transmitters, and ultrasonic waves transmitted by the plurality of ultrasonic transmitters may have different codes.

### BRIEF DESCRIPTION OF THE DRAWINGS

The present disclosure is further illustrated in terms of exemplary embodiments. These exemplary embodiments are described in detail with reference to the drawings. These embodiments are not limiting, and in these embodiments, the same numbering denotes the same structure, wherein:

FIG. 1 is a schematic diagram illustrating exemplary blocks of a human body posture recognition system according to some embodiments of the present disclosure;

FIG. 2 is a schematic diagram illustrating ultrasonic transmitters and ultrasonic receivers distributed on two opposite limbs corresponding to elbow joints or knee joints of a user according to some embodiments of the present disclosure;

FIG. 3 is a flowchart illustrating an exemplary process for recognizing a human posture according to some embodiments of the present disclosure;

FIG. 4 is a flowchart illustrating an exemplary process for recognizing a human posture according to some embodiments of the present disclosure;

FIG. 5 is a flowchart illustrating an exemplary process for recognizing a human posture according to some other embodiments of the present disclosure;

FIG. 6A is a schematic diagram illustrating recognizing a human posture according to some other embodiments of the present disclosure;

FIG. 6B is a side view illustrating one ultrasonic transmitter and two ultrasonic receivers distributed on two opposite limbs corresponding to an elbow joint or a knee joint according to some embodiments of the present disclosure;

FIG. 6C is a top view illustrating one ultrasonic transmitter and two ultrasonic receiver distributed on two opposite limbs corresponding to the elbow joint or the knee joint according to some embodiments of the present disclosure;

FIG. 7 is a schematic diagram illustrating an exemplary placement location of an ultrasonic sensor according to other embodiments of the present disclosure;

FIG. 8 is a schematic diagram illustrating an exemplary placement location of another ultrasonic sensor according to other embodiments of the present disclosure;

FIG. 9 is a schematic diagram illustrating ultrasonic transmitters and ultrasonic receivers distributed on upper arms and torso sites of the user according to some embodiments of the present disclosure;

FIG. 10 is a schematic diagram illustrating an exemplary placement location of the ultrasonic sensor according to some embodiments of the present disclosure;

FIG. 11 is a flowchart illustrating an exemplary process for determining an angle of the upper arm relative to the torso site according to some other embodiments of the present disclosure;

FIG. 12 is a flowchart illustrating an exemplary process for recognizing a motion state of the upper arm relative to the torso site according to some embodiments of the present disclosure;

FIG. 13 is a schematic diagram illustrating an exemplary placement location of the ultrasonic sensor according to other embodiments of the present disclosure;

FIG. 14 is a schematic diagram illustrating an exemplary three-point locationing manner according to some embodiments of the present disclosure;

FIG. 15 is a schematic diagram illustrating ultrasonic transmitters and ultrasonic receivers distributed on thighs and calves according to some embodiments of the present disclosure;

FIG. 16 is a flowchart illustrating an exemplary process for recognizing a posture of a leg of the user according to some embodiments of the present disclosure; and

FIG. 17 is a schematic diagram illustrating realizing time-sharing multiplexing based on the ultrasonic sensor according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios based on the accompanying drawings without creative labor. Unless obviously obtained from the context or the context illustrates otherwise, the same numeral in the drawings refers to the same structure or operation.

It should be understood that as used herein, the terms “system”, “device”, “unit” and/or “module” are used herein as a way to distinguish between different components, elements, parts, sections, or assemblies at different levels. However, the words may be replaced by other expressions if other words accomplish the same purpose.

As shown in the present disclosure and the claims, unless the context clearly suggests an exception, the words “one,” “a”, “an”, “one kind”, and/or “the” do not refer specifically to the singular, but may also include the plural. Generally, the terms “including” and “comprising” suggest only the inclusion of clearly identified steps and elements that do not constitute an exclusive list, and the method or apparatus may also include other steps or elements.

Flowcharts are used in this application to illustrate operations performed by a system according to embodiments of this application. It should be appreciated that the preceding or following operations are not necessarily performed in an exact sequence. Instead, steps can be processed in reverse order or simultaneously. Also, it is possible to add other operations to these processes or remove a step or steps from them.

Embodiments of the present disclosure provide a human body posture recognition system. In some embodiments, the human posture recognition system may include at least one group of ultrasonic sensors and a processor. In some embodiments, each group of ultrasonic sensors may include an ultrasonic transmitter configured to transmit ultrasonic waves and an ultrasonic receiver configured to receive the ultrasonic waves, and the ultrasonic transmitter and the ultrasonic receiver are located at different parts of a user's body. For example, the ultrasonic transmitter and the ultrasonic receiver may be located at an upper arm and a lower arm, respectively, on two sides of an elbow joint of the user. As another example, the ultrasonic transmitter and the ultrasonic receiver may be located at a thigh and a calf, respectively, on two sides of a knee joint of the user. As another example, the ultrasonic transmitter and the ultrasonic receiver may be located on the upper arm and a torso site. As another example, the ultrasonic transmitter and the ultrasonic receiver may be located on legs of the user, respectively. The processor may be configured to recognize a posture of the user based on location information of the ultrasonic transmitter and the ultrasonic receiver, information of the ultrasonic waves transmitted by the ultrasonic transmitter, and information of the ultrasonic waves received by the ultrasonic receiver. The human body posture recognition system provided in the embodiments of the present disclosure provides the ultrasonic transmitter and the ultrasonic receiver at different parts of the user's body, and the processor may obtain a distance between the ultrasonic transmitter and the ultrasonic receiver at different parts of the user's body based on the information of the ultrasonic waves transmitted by the ultrasonic transmitter and the information of the ultrasonic waves received by the ultrasonic receiver. Further, locations of the ultrasonic transmitter and the ultrasonic receiver on the user's body are fixed, and a spatial coordinate system may be established with any point of a human body in a standing posture as an origin to obtain the location information (e.g., three-dimensional coordinates) of the ultrasonic transmitter and the ultrasonic receiver at different parts of the user's body. When the user exercises, the processor may determine a posture of the user's limb and a posture of the limb relative to a torso site of the user based on the location information of the ultrasonic transmitter and the ultrasonic receiver, the information of the ultrasonic waves transmitted by the ultrasonic transmitter, and the information of the ultrasonic waves received by the ultrasonic receiver, so as to recognize a posture of the user's whole body. The human body posture recognition system provided in the present disclosure can improve the accuracy of recognizing the user's posture by designing a location and/or count of ultrasonic transmitters and ultrasonic receivers in an ultrasonic sensor. In addition, the human body posture recognition system provided in the present disclosure can set up different counts of ultrasonic sensors and/or at different locations for different joints or parts of human, henceforth improving the accuracy of the posture of the user obtained.

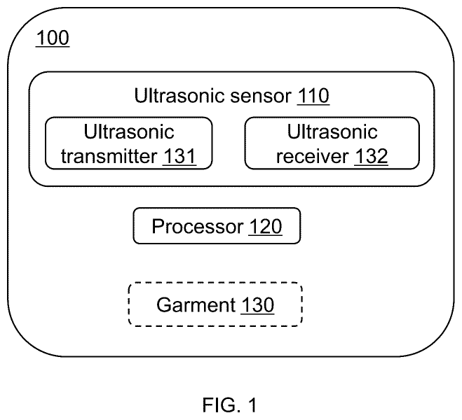

FIG. 1 is a schematic diagram illustrating exemplary blocks of a human body posture recognition system according to some embodiments of the present disclosure.

A human body posture recognition system 100 refers to a system for recognizing a posture of a body of human. For example, when a posture of the body changes, for example, when a user bends an arm, the human body posture recognition system 100 may recognize that the user's arm performs a bending action based on a bending situation of the user's arm. As shown in FIG. 1, the human body posture recognition system 100 may include at least one group of ultrasonic sensors 110 and a processor 120. In some embodiments, the human body posture recognition system 100 may include a plurality of groups of ultrasonic sensors 110, and the plurality of groups of ultrasonic sensors 110 may be located at different parts of the user. For example, the plurality of ultrasonic sensors may be located at any one or more of the user's limbs (e.g., arms, legs, etc.), torso sites (e.g., a shoulder portion, a chest portion, a back portion, a lumbar portion, etc.), head, etc.

In some embodiments, the ultrasonic transmitter 131 and the ultrasonic receiver 132 are located at different parts of the user's body. For example, the ultrasonic transmitter and the ultrasonic receiver may be located at an upper arm and a lower arm, respectively, on two sides of an elbow joint of the user. As another example, the ultrasonic transmitter and the ultrasonic receiver may be located at a thigh and a calf, respectively, on two sides of a knee joint of the user. As another example, the ultrasonic transmitter and the ultrasonic receiver may be located at an upper arm and a torso site of the user. As another example, the ultrasonic transmitter and the ultrasonic receiver may be located at legs of the user, respectively. In some embodiments, the each group of ultrasonic sensors 110 may include at least one ultrasonic transmitter 131 and at least one ultrasonic receiver 132. For example, the each group of ultrasonic sensors 110 may include one ultrasonic transmitter 131 and a plurality of ultrasonic receivers 132. As another example, the each group of ultrasonic sensors 110 may include a plurality of ultrasonic transmitters 131 and one ultrasonic receiver 132. As another example, the each group of ultrasonic sensors 110 may include a plurality of ultrasonic transmitters 131 and a plurality of ultrasonic receivers 132. In some embodiments, when the human body posture recognition system 100 includes a plurality of groups of ultrasonic sensors 110, counts of ultrasonic transmitters 131 and ultrasonic receivers 132 corresponding to the plurality of groups of ultrasonic sensors 110 may be the same or different. For example, a count of ultrasonic transmitters 131 and ultrasonic receivers 132 distributed on an upper arm and a lower arm on two sides of the elbow joint of the user is less than a count of ultrasonic transmitters 131 and ultrasonic receivers 132 distributed on an inner side of the upper arm and a torso site of the user.

The ultrasonic transmitter 131 refers to a device capable of transmitting ultrasonic waves. The ultrasonic transmitter 131 may convert an electrical signal into ultrasonic waves and transmit the ultrasonic waves. In some embodiments, the ultrasonic transmitter 131 may include but is not limited to, a magneto strictive transmitter, a piezoelectric ultrasonic transmitter, a micromechanical ultrasonic transmitter, or the like.

The ultrasonic receiver 132 refers to a device capable of receiving ultrasonic waves. The ultrasonic receiver 132 may receive ultrasonic waves and convert the ultrasonic waves into an electrical signal. In some embodiments, the ultrasonic receiver 132 may include but is not limited to, a magneto strictive receiver, a piezoelectric ultrasonic receiver, a micromechanical ultrasonic receiver, or the like.

The processor 120 may be configured to process information and/or data related to the human body posture recognition system 100, for example, the processor 120 may process location information of the ultrasonic transmitter and the ultrasonic receiver, information of ultrasonic waves transmitted by the ultrasonic transmitter, and information of ultrasonic waves received by the ultrasonic receiver, or the like. The processor 120 may process data, information, and/or processing results obtained from other devices or system components and execute program instructions to perform one or more of the functions described in the present disclosure based on the data, information, and/or processing results. By way of example only, the processor 120 may include a central processing unit (CPU), an application-specific integrated circuit (ASIC), an application-specific instruction processor (ASIP), a reduced instruction set computer (RISC), a microprocessor, or the like, or any combination of the above.

In some embodiments, the processor 120 may be configured to recognize a posture of the user based on the location information of the ultrasonic transmitter and the ultrasonic receiver, the information of the ultrasonic waves transmitted by the ultrasonic transmitter, and the information of the ultrasonic waves received by the ultrasonic receiver.

The location information of the ultrasonic transmitter and the ultrasonic receiver refers to information of a spatial distribution of the ultrasonic transmitter and the ultrasonic receiver on a body of the user. For example, a location of the ultrasonic transmitter on the body of the user, a location of the ultrasonic receiver on the body of the user, etc. The location information of the ultrasonic transmitter and the ultrasonic receiver may be represented by coordinates, etc. For example, locations of the ultrasonic transmitter and the ultrasonic receiver on the body of the user are fixed, and a spatial coordinate system may be established with any point of the body in a standing posture as an origin to obtain the location information (e.g., 3D coordinates) of the ultrasonic transmitter and the ultrasonic receiver at different parts of the user's body. It should be noted that the location information of the ultrasonic transmitter and the ultrasonic receiver may be fixed, or may be adapted according to the user's age, gender, height, weight, and body type.

The information of the ultrasonic waves transmitted by the ultrasonic transmitter refers to information related to a process in which the ultrasonic transmitter transmits the ultrasonic waves. For example, a time at which the ultrasonic transmitter transmits the ultrasonic waves, a frequency at which the ultrasonic transmitter transmits the ultrasonic waves, a code at which the ultrasonic transmitter transmits the ultrasonic waves, or the like. The information of the ultrasonic waves received by the ultrasonic receiver refers to information related to a process in which the ultrasonic receiver receives the ultrasonic waves. For example, a time at which the ultrasonic receiver receives the ultrasonic waves, a frequency at which the ultrasonic receiver receives the ultrasonic waves, a code at which the ultrasonic receiver receives the ultrasonic waves, or the like.

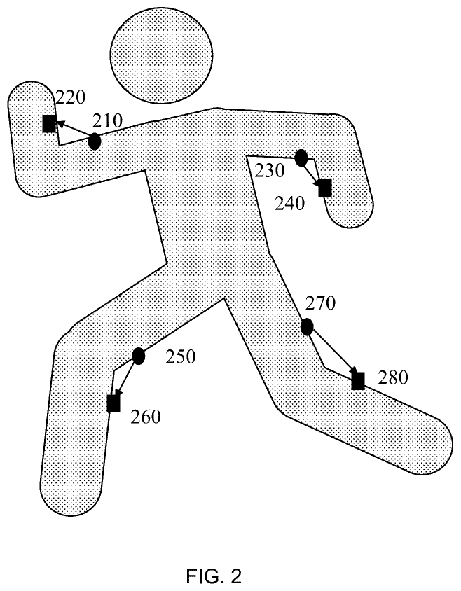

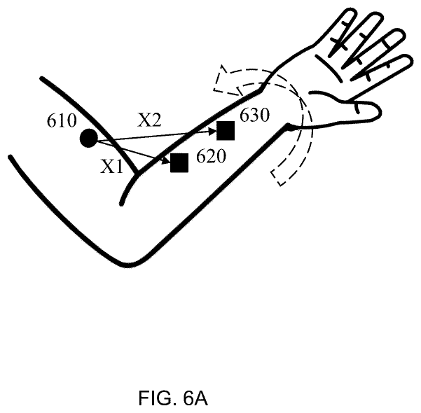

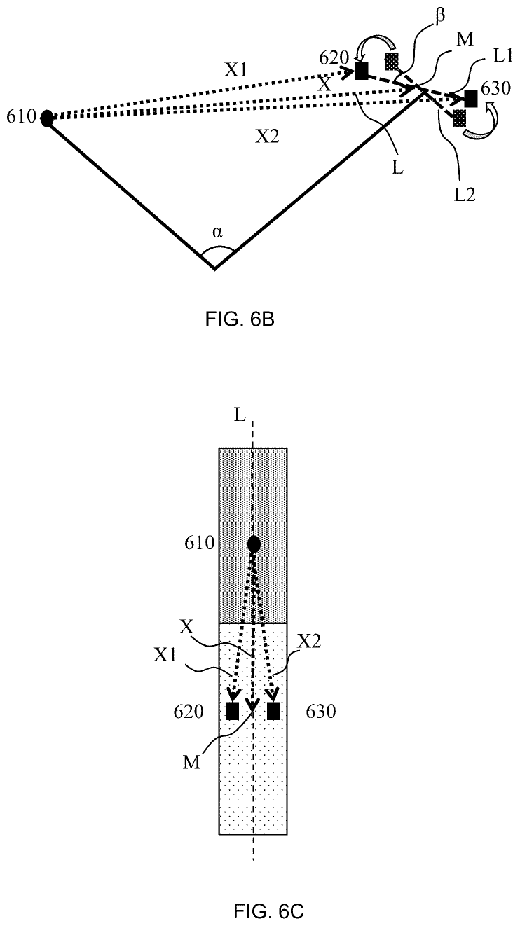

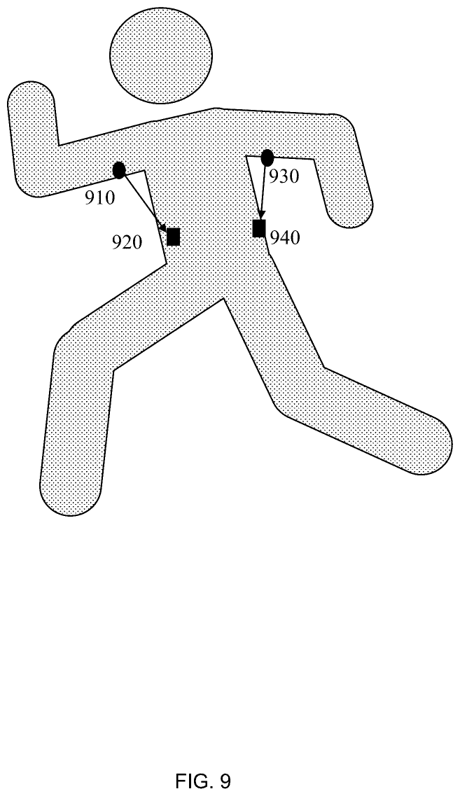

In some embodiments, recognition of the posture of the user may be achieved by designing a placement location of the ultrasonic transmitter and the ultrasonic receiver in the ultrasonic sensor. For example, by arranging the ultrasonic transmitter and the ultrasonic receiver at the user's right lower arm and right upper arm, respectively, the processor 120 may recognize a bending angle between two opposite limbs corresponding to an elbow joint or a knee joint of the user based on the location information of the ultrasonic transmitter and the ultrasonic receiver, the information of the ultrasonic waves transmitted by the ultrasonic transmitter, and the information of the ultrasonic waves received by the ultrasonic receiver. As another example, arranging the ultrasonic receiver at an upper arm and the ultrasonic transmitter at a torso site, and the processor 120 may recognize a posture of the upper arm relative to the torso site (e.g., the upper arm swings back and forth, up and down, and left and right relative to the torso location) based on the location information of the ultrasonic transmitter and the ultrasonic receiver, the information of the ultrasonic waves transmitted by the ultrasonic transmitter, and the information of the ultrasonic waves received by the ultrasonic receiver. More information about recognizing the bending angle between the two opposite limbs corresponding to the elbow joint or the knee joint and recognizing the posture of the upper arm relative to the torso site can be found in FIG. 2 to FIG. 5, FIG. 6A, FIG. 6B, FIG. 6C, FIG. 9 to FIG. 14 and the related descriptions thereof.

In some embodiments, the human body posture recognition system 100 may further include a garment 130, the garment 130 may be for the user to wear, with the at least one group of ultrasonic sensors 110 integrated into the garment. In some embodiments, the garment 130 may include any one or more of a shirt, pants, jumpsuit, etc. In some embodiments, the ultrasonic sensor 110 may be integrated into the garment in a variety of ways. For example, the ultrasonic sensor may be sewn to the garment via a sewing thread such as cotton thread, nylon thread, or the like. For example, the ultrasonic sensor 110 may be sewn to the garment via a sewing thread such as cotton thread, nylon thread, or the like. As another example, the ultrasonic sensor 110 may be set with the garment via a removable structure such as a snap, Velcro, or the like. In some embodiments, the garment 130 may be a single-layer structure, and the ultrasonic sensor 110 may be disposed on an upper surface or a lower surface of the single-layer structure. In some embodiments, the garment 130 may be a multi-layer structure, and the ultrasonic sensor 110 may be disposed on an upper surface, a lower surface, or between two adjacent layers of the garment 130.

In some embodiments, the processor 120 may be integrated into the garment 130. For example, the processor 120 may be provided on the garment 130 by sewing, bonding, or the like. In some embodiments, the processor 120 may be provided separately from the garment 130. For example, the processor 120 may be in communication with the garment 130 or the ultrasonic sensor 110 disposed on the user's body over a wired network and/or a wireless network. Exemplary wired networks may include cable networks, fiber optic networks, or the like. Exemplary wireless networks may include wireless local area networks (WLANs), Bluetooth networks, global system for mobile communications (GSM) networks, or the like.

In some embodiments, the ultrasonic sensor in the human body posture recognition system 100 may also be directly disposed on the user's body. For example, the ultrasonic transmitter and the ultrasonic receiver corresponding to the ultrasonic sensor may be secured to the user's body or clothing by means of a fastening member (e.g., a strap structure, a pin, a carabiner), glue, or an adhesive member.

In order to improve the accuracy of the human body posture recognition system 100 in recognizing the posture of the user, in some embodiments, the ultrasonic sensor 110 may be provided at different parts of the user's body, furthermore, an ultrasonic transmitter 131 and an ultrasonic receiver 132 correspond to each group of ultrasonic sensors 110 are distributed at different parts of the user's body, and the following illustrates a posture of different parts in conjunction with a distribution of the ultrasonic transmitter 131 and the ultrasonic receiver 132.

In some embodiments, the ultrasonic transmitter and the ultrasonic receiver may be disposed at two opposite limbs corresponding to the user's elbow joint or knee joint, respectively, in order to recognize a motion posture of the user's arm or leg. For example, recognizing a bending angle or a rotation angle, etc., between the two opposite limbs corresponding to the user's elbow joint or knee joint. Limbs on two sides corresponding to the elbow joint refer to an upper arm and a lower arm, and limbs on two sides corresponding to the knee joint refer to a thigh and a calve. For example, one of the ultrasonic transmitter and the ultrasonic receiver is located at the upper arm and the other is located at the lower arm. As another example, one of the ultrasonic transmitter and the ultrasonic receiver is located at the thigh and the other is located at the calve. In some embodiments, when the ultrasonic sensor is integrated into the garment, the ultrasonic transmitter and the ultrasonic receiver are arranged at a sleeve or a trouser leg of the garment, such that when the user wears the garment, the ultrasonic transmitter and the ultrasonic receiver may be respectively located at the two opposite limbs corresponding to the user's elbow joint or knee joint.

FIG. 2 is a schematic diagram illustrating ultrasonic transmitters and ultrasonic receivers distributed on two opposite limbs corresponding to elbow joints or knee joints of a user according to some embodiments of the present disclosure; As shown in FIG. 2, an ultrasonic transmitter 210 and an ultrasonic receiver 220 are located at two opposite limbs corresponding to the user's elbow joint, where the ultrasonic transmitter 210 is located at the user's left upper arm and the ultrasonic receiver 220 is located at the user's left lower arm; an ultrasonic transmitter 230 and an ultrasonic receiver 240 are located at two opposite limbs corresponding to the user's elbow joint, where the ultrasonic transmitter 230 is located at the user's right upper arm and the ultrasonic receiver 240 is located at the user's right lower arm; an ultrasonic transmitter 250 and an ultrasonic receiver 260 are located at two opposite limbs corresponding to the user's knee joint, where the ultrasonic transmitter 250 is located at the user's left thigh and the ultrasonic receiver 260 is located at the user's left calf; and an ultrasonic transmitter 270 and an ultrasonic receiver 280 are located at two opposite limbs corresponding to the user's knee joint, where the ultrasonic transmitter 270 is located at the user's right thigh and the ultrasonic receiver 280 is located at the user's right calf.

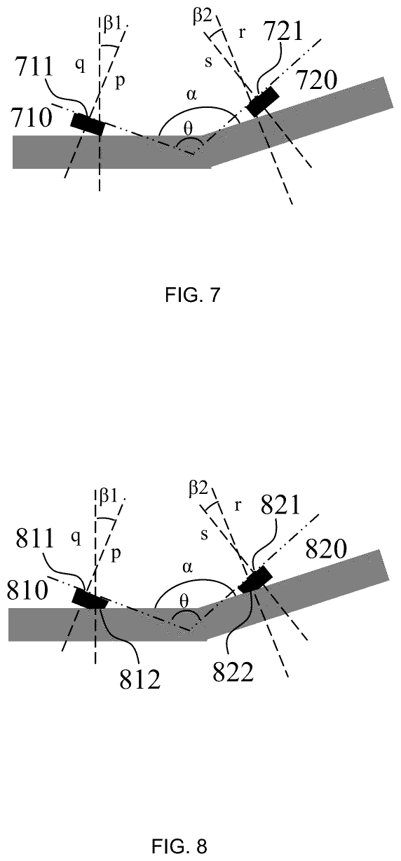

Placement locations of an ultrasonic sensor shown in FIG. 2 are for example only, and it will be appreciated that locations of the ultrasonic transmitter and the ultrasonic receiver, as shown in FIG. 2, may be interchangeable. For example, the ultrasonic transmitter 210 is located at the user's left lower arm, and the ultrasonic receiver 220 is located at the user's left upper arm. As another example, the ultrasonic transmitter 270 is located at the user's right calf and the ultrasonic receiver 280 is located at the user's thigh. More information about the placement locations of the ultrasonic sensor can be found in FIG. 7 and FIG. 8 and their descriptions.

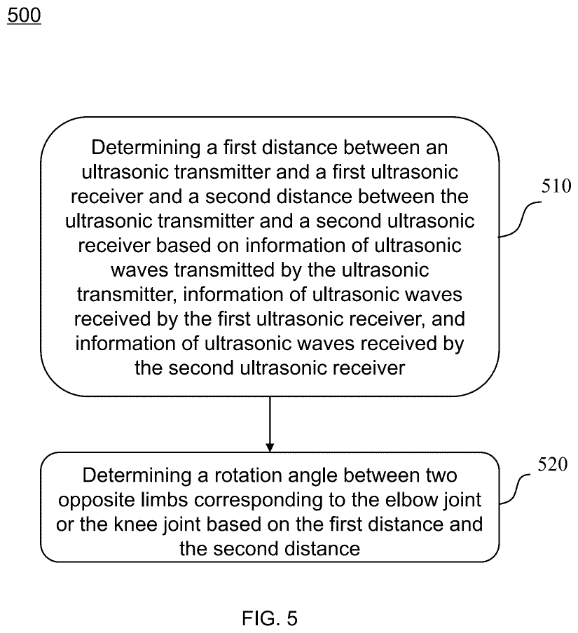

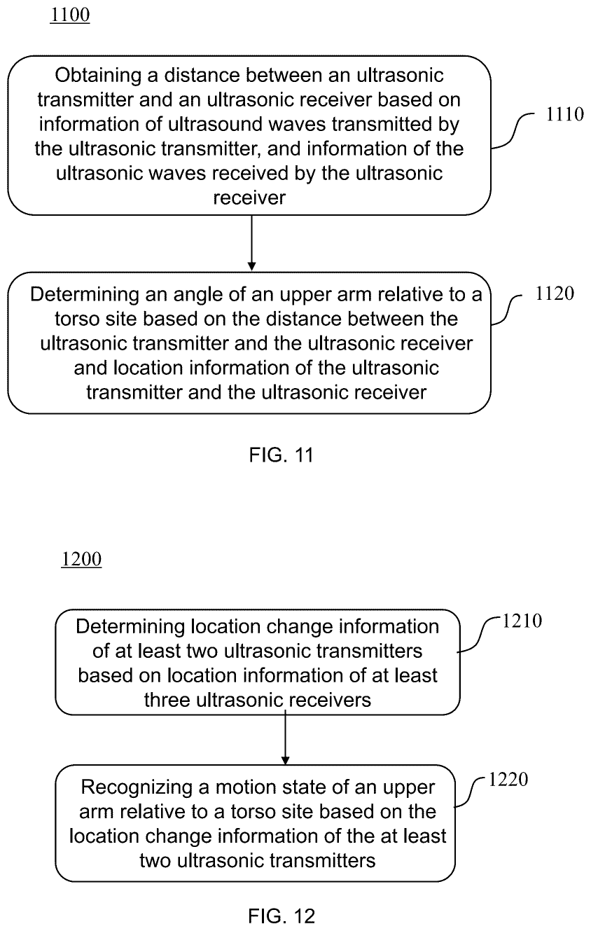

In order to further improve the recognition accuracy of the human body posture recognition system, to reduce a measurement dead space, and to prevent the effect of human body occlusion, each group of ultrasonic sensors may include a plurality of ultrasonic transmitters or a plurality of ultrasonic receivers. For example, one ultrasonic transmitter is provided at the user's left lower arm, and two ultrasonic receivers are provided at the user's left upper arm. As another example, three ultrasonic transmitters are provided at the user's left lower arm and three ultrasonic receivers are provided at the user's left upper arm. More information about setting up a plurality of ultrasonic transmitters and a plurality of ultrasonic receivers can be found elsewhere in the present disclosure, e.g., FIG. 5 to FIG. 6C, FIG. 11 to FIG. 14, and their related descriptions thereof.

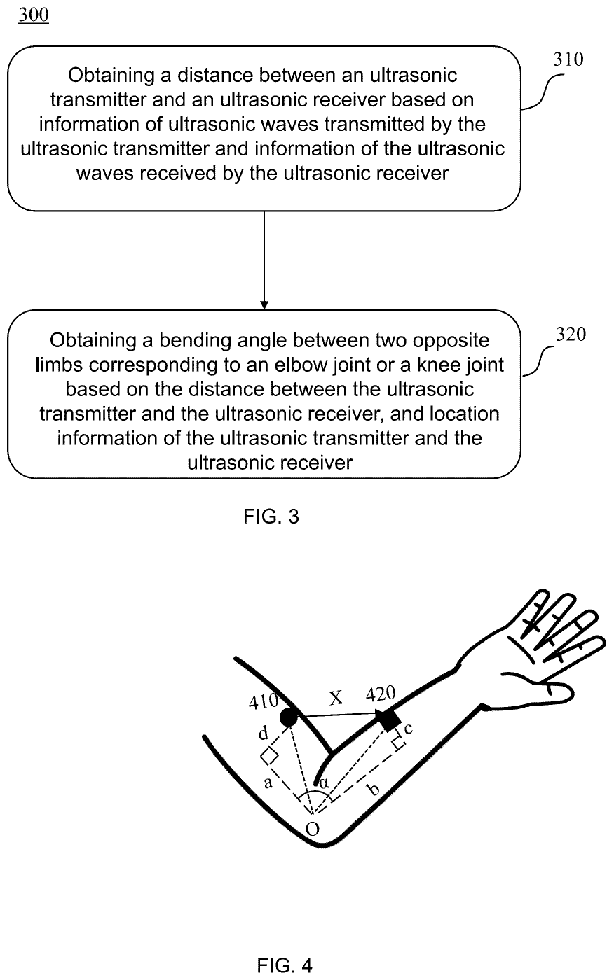

FIG. 3 is a flowchart illustrating an exemplary process for recognizing a posture of a user according to some embodiments of the present disclosure. In some embodiments, a process 300 may be executed by the processor 120. As shown in FIG. 3, the process 300 may include following steps.

Step 310, obtaining a distance between an ultrasonic transmitter and an ultrasonic receiver based on information of ultrasonic waves transmitted by the ultrasonic transmitter and information of the ultrasonic waves received by the ultrasonic receiver.

In some embodiments, the processor may obtain the distance between the ultrasonic transmitter and the ultrasonic receiver based on a time at which the ultrasonic transmitter transmits the ultrasonic waves and a time at which the ultrasonic receiver receives the ultrasonic waves. Using as an example that the ultrasonic transmitter and the ultrasonic receiver are distributed on an upper arm and a lower arm on both sides of an elbow joint, an ultrasonic transmitter 410 is located at the upper arm of the user and an ultrasonic receiver 420 is located at the lower arm of the user, as illustrated in FIG. 4. The ultrasonic transmitter 410 transmits ultrasonic waves that may be received by the ultrasonic receiver 420 when a human body posture recognition system is in operation. Specifically, the ultrasonic transmitter 410 transmits the ultrasonic waves at a time t1 and the ultrasonic receiver 420 receives the ultrasonic waves at a time t2, and the processor may, based on a difference Δt between t1 and t2, a velocity of propagation of the ultrasonic waves in the air C (approximately 340 m/s at 1 standard atmosphere and 15° C.), multiply Δt by C, and determine a multiplication result as a distance X between the ultrasonic transmitter 410 and the ultrasonic receiver 420.

Step 320, obtaining a bending angle between two opposite limbs corresponding to an elbow joint or a knee joint based on the distance between the ultrasonic transmitter and the ultrasonic receiver, and location information of the ultrasonic transmitter and the ultrasonic receiver.

The bending angle between the two opposite limbs corresponding to the elbow joint or the knee joint refers to a bending angle between a left (right) lower arm and a left (right) upper arm or a bending angle between a left (right) calve and a left (right) thigh. Taking as an example that the ultrasonic transmitter and the ultrasonic receiver are distributed on the upper arm and the lower arm on both sides of the elbow joint, as shown in FIG. 4, a point O shown in FIG. 4 may be regarded as a location where the elbow joint is located, and an angle α may be regarded as a bending angle (i.e., an angle between the upper arm and the lower arm). It should be noted that here the bending angle may be regarded as an angle between an extension direction of the upper arm and an extension direction of the lower arm, and the point O may be regarded as an intersection between the extension direction of the upper arm and the extension direction of the lower arm.

In some embodiments, the processor may obtain a bending angle α between the two opposite limbs corresponding to the elbow joint or the knee joint, based on the distance between the ultrasonic transmitter and the ultrasonic receiver and the location information of the ultrasonic transmitter and the ultrasonic receiver, by means of a formula (1):

$$α = Arc Tan ( d / a ) + Arc Tan ( c / b ) + Arc Cos ( \frac{{a}^{2} + {d}^{2} + {b}^{2} + {c}^{2} - {x}^{2}}{2 \sqrt{{a}^{2} + {d}^{2}} \sqrt{{b}^{2} + {c}^{2}}} ) . \qquad ( 1 )$$

Where, c denotes a length value that may characterize a dimension at the user's lower arm where the ultrasonic transmitter is located, and d denotes a length value that may characterize a dimension at the user's upper arm where the ultrasonic receiver is located. A value of c or d is positively correlated with a dimension of the user's arm. For example, the thicker the user's upper arm or lower arm, the larger c or d. For exemplary illustration only, a perimeter of a cross-sectional shape of the lower arm or the upper arm intercepted along a direction perpendicular to the extension direction of the lower arm or the upper arm and over a geometric center of the ultrasonic transmitter is used as the dimension at the user's lower arm or upper arm, and the cross-sectional shape may be approximated as an ellipse, with a radius of a long axis or a radius of a short axis of the ellipse being c or d. a may characterize a distance between a center point of the cross-sectional shape and a joint (e.g., the elbow joint, characterized by the point O in FIG. 4), and b may characterize a distance between a center of the cross-sectional shape and a joint (e.g., the elbow joint). a, b, c, and d are known parameters. c and d may be determined based on the dimension of the user's arm/leg, for example, c and d may be determined by measurement based on the dimension of the user's arm or leg. a and b may be determined based on the location information of the ultrasonic transmitter and the ultrasonic receiver, e.g., by establishing a three-dimensional spatial coordinate system using a certain location of a human body as an element, determining a and b based on the location information (coordinate information) of the ultrasonic transmitter and the ultrasonic receiver, coordinate information of the elbow joint (e.g., the point O) coordinate information, the dimension of the user's arm, or the like. An ultrasonic transmitter and an ultrasonic receiver corresponding to an ultrasonic sensor are set on two opposite limbs corresponding to the knee joint or the elbow joint of the user, and based on information of the ultrasonic sensor (e.g., the ultrasonic transmitter and the ultrasonic receiver), a bending angle between the two opposite limbs corresponding to the elbow joint or the knee joint may be determined to recognize a localized motion posture of the user's limbs. The human body posture recognition system is stable with a simpler algorithm, which can recognize a motion state of the user's limbs more accurately.

The motion state of the two opposite limbs corresponding to the elbow joint or the knee joint is not a bending motion centered on the elbow joint or the knee joint, and the motion state of the two opposite limbs corresponding to the joints may also be a rotation centered on the joints. For example, the lower arm may rotate relative to the upper arm with the elbow joint as a center. In order to more accurately recognize the posture of the user's limbs, in some embodiments, by arranging the ultrasonic transmitter and the ultrasonic receiver on the two opposite limbs corresponding to the elbow joint or the knee joint, respectively, it is also possible to recognize a rotation angle between the two opposite limbs corresponding to the elbow joint or the knee joint. FIG. 5 is a flowchart illustrating an exemplary process for recognizing a posture of a user according to other embodiments of the present disclosure. In some embodiments, a process 500 may be executed by the processor 120. As shown in FIG. 5, the process 500 may include following steps.

Step 510, determining a first distance between an ultrasonic transmitter and a first ultrasonic receiver and a second distance between the ultrasonic transmitter and a second ultrasonic receiver based on information of ultrasonic waves transmitted by the ultrasonic transmitter, information of ultrasonic waves received by the first ultrasonic receiver, and information of ultrasonic waves received by the second ultrasonic receiver.

In order to further improve the recognition accuracy of a human body posture recognition system and reduce a measurement dead space, each group of ultrasonic sensors may include a plurality of ultrasonic transmitters or a plurality of ultrasonic receivers. For example, each group of ultrasonic sensors may include at least two ultrasonic transmitters and one ultrasonic receiver. As another example, each group of ultrasonic sensors may include at least two ultrasonic transmitters and at least two ultrasonic receivers. As another example, each group of ultrasonic sensors may include one ultrasonic transmitter and at least two ultrasonic receivers. In determining a rotation angle between two opposite limbs corresponding to an elbow joint or a knee joint, each group of ultrasonic sensors may include a plurality of ultrasonic transmitters or a plurality of ultrasonic receivers. For illustrative purposes, the following will be an example when each group of ultrasonic sensors may include one ultrasonic transmitter and at least two ultrasonic receivers.

In some embodiments, each group of ultrasonic sensors includes an ultrasonic transmitter and at least two ultrasonic receivers, the at least two ultrasonic receivers including a first ultrasonic receiver and a second ultrasonic receiver. As shown in FIG. 6A, each group of ultrasonic sensors includes an ultrasonic transmitter 610, a first ultrasonic receiver 620, and a second ultrasonic receiver 630.

The first distance refers to a distance between the ultrasonic transmitter and the first ultrasonic receiver. A distance between the ultrasonic transmitter 610 and the first ultrasonic receiver 620 is X1, as shown in FIG. 6A. The second distance refers to a distance between the ultrasonic transmitter and the second ultrasonic receiver. A distance between the ultrasonic transmitter 610 and the second ultrasonic receiver 630 is X2, as shown in FIG. 6A.

In some embodiments, a processor may determine, based on a time at which the ultrasonic transmitter transmits ultrasonic waves, a time at which the first ultrasonic receiver receives the ultrasonic waves, and a time at which the second ultrasonic receiver receives the ultrasonic waves, the first distance between the ultrasonic transmitter and the first ultrasonic receiver and the second distance between the ultrasonic transmitter and the second ultrasonic receiver. Taking the ultrasonic transmitter and the ultrasonic receiver distributed on an upper arm and a lower arm located on two sides of an elbow joint as an example, as shown in FIG. 6A, the ultrasonic transmitter 610 is located at the upper arm of the user, and the first ultrasonic receiver 620 and the second ultrasonic receiver 630 are located at the lower arm of the user, when the human body posture recognition system is in operation, the ultrasonic transmitter 610 transmits ultrasonic waves that may be received by the first ultrasonic receiver 620 and the second ultrasonic receiver 630, specifically, the ultrasonic transmitter 610 transmits the ultrasonic waves at a time t1, the first ultrasonic receiver 620 receives the ultrasonic waves at a time t2, and the second ultrasonic receiver 630 receives the ultrasonic waves at a time t3, and based on a difference Δt12 between t1 and t2 and a difference Δt13 between t1 and t3, and the ultrasonic waves propagate through the air at a speed of C (1 standard atmospheric pressure and about 340 m/s at 15° C.), the processor may multiply Δt12, Δt13 with C, respectively, and determine multiplication results as the distance X1 between the ultrasonic transmitter 610 and the first ultrasonic receiver 620 and the distance X2 between the ultrasonic transmitter 610 and the second ultrasonic receiver 630.

Taking a right arm of a human body as an example, when the arm is normally straightened, the lower arm has not rotated relative to the upper arm, and due to the nature of the elbow joint of the human body, the lower arm normally rotates in a counterclockwise direction (in a direction indicated by the arrow shown in FIG. 6A) in an extension direction of the arm (in a direction from the lower arm to a hand). Based on this, in some embodiments, the first ultrasonic receiver 620 may be disposed at a location on a same side of the user's lower arm and palm, and the second ultrasonic receiver 630 may be located at a location on a same side of the user's lower arm and back of a hand, so as to more accurately recognize a rotation angle of the lower arm relative to the upper arm of the user.

Step 520, determining a rotation angle between two opposite limbs corresponding to the elbow joint or the knee joint based on the first distance and the second distance.

The rotation angle between the two opposite limbs corresponding to the elbow joint refers to a rotation angle of the lower arm relative to the upper arm. The rotation angle between the two opposite limbs corresponding to the knee joint refers to a rotation angle of the calve relative to the thigh.

For greater clarity, FIG. 6B and FIG. 6C are described below in conjunction. FIG. 6B is a side view illustrating one ultrasonic transmitter and two ultrasonic receivers distributed on two opposite limbs corresponding to an elbow joint or a knee joint according to some embodiments of the present disclosure, and FIG. 6C is a top view illustrating one ultrasonic transmitter and two ultrasonic receivers distributed on two opposite limbs corresponding to an elbow joint or a knee joint according to some embodiments of the present disclosure.

As shown in FIG. 6B and FIG. 6C, in some embodiments, the first ultrasonic receiver 620 and the second ultrasonic receiver 630 may be set approximately symmetrically to a straight line (e.g., a straight line L illustrated in FIG. 6B and FIG. 6C) that is parallel to an extension direction of an upper arm and passes over the ultrasonic transmitter. When the arm is normally straightened and the small arm has not rotated relative to the large arm, the first distance X1 and the second distance X2 shown in FIG. 6B or FIG. 6C may be regarded as approximately equal, and at this time the first ultrasonic receiver 620 and the second ultrasonic receiver 630 are connected by a first straight line L1.

When the lower arm rotates with respect to the upper arm, the first ultrasonic receiver 620 and the second ultrasonic receiver 630 also rotate with respect to the ultrasonic transmitter 610, and at this time, a line connecting the first ultrasonic receiver 620 and the second ultrasonic receiver 630 is a second straight line L2. When the user's lower arm and the upper arm change from a straight state to a state in which the lower arm rotates, an angle β formed between the first straight line L1 and the second straight line L2 may be regarded as a rotation angle of the lower arm relative to the upper arm. When the lower arm rotates relative to the upper arm, the first distance X1 and the second distance X2 may change. For example, as shown in FIG. 6B or FIG. 6C, when the lower arm rotates relative to the upper arm toward a side of the arm on which the first ultrasonic receiver 620 is located (i.e., in a direction of a circular arrow shown in FIG. 6B or FIG. 6C), the first distance X1 becomes larger and the second distance X2 becomes smaller, resulting in the first distance X1 being larger than the second distance X2. When the lower arm rotates relative to the upper arm toward a side of the arm on which the second ultrasonic receiver 630 is located, the first distance X1 becomes smaller and the second distance X2 becomes larger, resulting in the first distance X1 being smaller than the second distance X2.

In some embodiments, the processor may determine a rotation angle β between two opposite limbs corresponding to an elbow joint or a knee joint based on the first distance and the second distance, which may be derived from a formula (2):

$$β ∝ \pm ( X 1 - X 2 ) . \qquad ( 2 )$$

where, ± denotes a rotation direction, + denotes that the lower arm rotates relative to the upper arm toward a side of the arm on which the first ultrasonic receiver 620 is located, and − denotes that the lower arm rotates relative to the upper arm toward a side of the arm on which the second ultrasonic receiver 630 is located.

It is important to note that the rotation angle β determined through the formula (2) is a relative quantity used only to characterize a rotation degree of a lower arm or calve, and is not a precise rotation angle between the two opposite limbs corresponding to the elbow joint or the knee joint.

In some embodiments, when a count of ultrasonic receivers in a group of ultrasonic sensors is two, the processor may determine a bending angle between two opposite limbs corresponding to the elbow joint or the knee joint based on the first distance and the second distance. As shown in FIG. 6B and FIG. 6C, the processor may obtain a distance X between a midpoint M of a line that connects the first ultrasonic receiver 620 and the second ultrasonic receiver 630 and the ultrasonic transmitter 610 based on the first distance X1 and the second distance X2 by adding the first distance and the second distance together and dividing the first distance and the second distance by two, and a bending angle α of the elbow joint may be determined based on the formula (1). More information about the formula (1) can be found in FIG. 3 and the descriptions thereof. It should be noted that the calculation of X herein is approximately equal since a distance between the first ultrasonic receiver 620 and the second ultrasonic receiver 630 is much smaller than a distance between the ultrasonic transmitter 610 and the first ultrasonic receiver 620 or between the ultrasonic transmitter 610 and the second ultrasonic receiver 630, so in this case, a calculation result X may be approximated to be equal to a distance between the ultrasonic transmitter and the ultrasonic receiver.

The ultrasonic sensor has a limited operation range, e.g., the ultrasonic transmitter may only transmit ultrasonic waves in a certain direction and the ultrasonic receiver may only receive the ultrasonic waves in a certain direction. The operation range of the ultrasonic sensor may be determined by a manufacturing process of the ultrasonic sensor to some extent. Therefore, it is necessary to understand the operation range of the ultrasonic sensor before designing a placement location of the ultrasonic sensor, the following may explain a structure of the ultrasonic transmitter and the ultrasonic receiver.

In some embodiments, the ultrasonic transmitter may include a vibration unit and a housing. The vibration unit may convert an electrical signal into ultrasonic waves. The housing may fix and support the ultrasonic transmitter, isolate the ultrasonic transmitter from the external environment, and so on. Additionally, the housing may be set up in a variety of ways to limit a transmission direction of the ultrasonic waves. For example, the housing may be provided with a hole portion to allow the ultrasonic waves to be transmitted from the hole portion. As another example, the housing may set a material (e.g., setting a type or a thickness of the material so that the material on that side is different from materials on other sides) or a structure (e.g., setting the structure to change, deform, or the like so that the structure on that side is different from structures on other sides) on one side to allow the ultrasonic waves to be transmitted from that side. In some embodiments, a side that transmits the ultrasonic waves may be referred to as an output end. For example, a side on the housing that has a hole portion with a different material than other sides may be referred to as the output end.

In some embodiments, the ultrasonic receiver may include a transducer unit and a housing. The transducer unit may convert ultrasonic waves into an electrical signal. The housing may fix and support the ultrasonic receiver, isolate the ultrasonic receiver from the external environment, and so on. Additionally, the housing of the ultrasonic receiver may be set to limit a receiving direction of ultrasonic waves with reference to the housing of the ultrasonic transmitter described above. For example, setting a side with a hole portion, setting a material or structure on a side, or the like. In some embodiments, a side that receives the ultrasonic waves may be referred to as a receiving end. For example, a side on the housing that has a hole portion with a different material than other sides may be referred to as the receiving end.

To ensure that the ultrasonic waves transmitted by the ultrasonic transmitter are always received by the ultrasonic receiver, in some embodiments, the ultrasonic transmitter and the ultrasonic receiver are disposed on inner sides of two opposite limbs corresponding to the elbow joint or the knee joint. When the joint elbow or the knee joint is in a bending state, two opposite lateral portions of the limb corresponding to the elbow joint or the knee joint refer to the inner sides of the two opposite limbs corresponding to the elbow joint or the knee joint. Since the lower arm in the arm may rotate relative to the upper arm, using the arm as an example, the inner sides of the two opposite limbs corresponding to the elbow joint may also be a part that the arm and the palm are on a same side when the arm is naturally straightened. FIG. 7 is a schematic diagram illustrating an ultrasonic transmitter and an ultrasonic receiver distributed on two opposite limbs corresponding to an elbow joint or a knee joint according to some embodiments of the present disclosure. As shown in FIG. 7, an ultrasonic transmitter 710 and an ultrasonic receiver 720 are disposed on inner sides of the two opposite limbs corresponding to the elbow joint or the knee joint.

Too small a distance between the ultrasonic transmitter and ultrasonic receiver may result in an output end of the ultrasonic transmitter being too close to a receiving end of the ultrasonic receiver, and result in a measured difference in change distance between the ultrasonic transmitter and ultrasonic receiver being too small, so a bending angle of the joint may not be accurately calculated. In order to improve the accuracy of a human body posture recognition system, in some embodiments, a distance between the ultrasonic transmitter and the ultrasonic receiver may be not less than 10 cm when a user straightens an arm or a leg. Preferably, the distance between the ultrasonic transmitter and the ultrasonic receiver may be not less than 15 cm when the user straightens the arms or the leg. Further preferably, the distance between the ultrasonic transmitter and the ultrasonic receiver may be not less than 20 cm when the user straightens the arm or the leg. In order to allow the ultrasonic transmitter and ultrasonic receiver to be distributed on the user's limb without interfering with the normal functioning of other parts of the user, in some embodiments, the distance between the ultrasonic transmitter and the ultrasonic receiver may be not greater than a length of the user's limb (e.g., the leg or the arm). It should be noted that different users have limbs with different lengths, and in practical application scenarios, adaptations may be made according to an actual situation of the user (e.g., a length of a limb).

When the ultrasonic sensor is integrated into a garment, in some embodiments, the distance between the ultrasonic transmitter and the ultrasonic receiver may be not greater than a length of a sleeve or trouser leg in the garment. In some embodiments, when the ultrasonic sensor is integrated into the garment and the garment is in a lay-flat and unfolded state, the distance between the ultrasonic transmitter and the ultrasonic receiver may be no less than 10 cm. Preferably, when the ultrasonic sensor is integrated into the garment and the garment is in a lay-flat and unfolded state, the distance between the ultrasonic transmitter and the ultrasonic receiver may be no less than 15 cm. Further preferably, when the ultrasonic sensor is integrated into the garment and the garment is in a lay-flat unfolded state, the distance between the ultrasonic transmitter and the ultrasonic receiver may be no less than 20 cm. Understandably, lengths of limbs of each individual may be different, and thus the garment may be designed for different users so that the ultrasonic sensor is arranged in a desired location. Children's limbs are typically shorter, for example, a length of an arm is typically greater than 30 cm and a length of a leg is typically greater than 60 cm for a child between the ages of 4 and 12 years old, and a distance between an ultrasonic transmitter and an ultrasonic receiver at a sleeve of a child's garment may be 10 cm-30 cm, and the distance between the ultrasonic transmitter and the ultrasonic receiver at the sleeve of the children's garment may be 20 cm-50 cm. Exemplarily, relative to the length of the children's limb, a distance between an ultrasonic transmitter and an ultrasonic receiver at an arm or a leg of an adult garment may be 20 cm-50 cm.

In some embodiments, the ultrasonic transmitter includes an output end for transmitting ultrasonic waves, with an output direction of the output end oriented toward an outer side of the garment, or away from a site at which the ultrasonic transmitter directly or indirectly contacts the user's limb. As shown in FIG. 7, illustrated with the ultrasonic transmitter 710 and the ultrasonic receiver 720 being located at a surface of the garment or the user's skin as an example, the ultrasonic transmitter 710 includes an output end 711, an output direction of the output end 711 is oriented toward an outer side of the garment or away from a site at which the ultrasonic transmitter 710 directly or indirectly contacts the user's limb. In some embodiments, the ultrasonic receiver includes a receiving end for receiving ultrasonic waves, with a receiving end oriented toward the outer side of the garment, or away from a site at which the ultrasonic receiver directly or indirectly contacts the user's limb. As shown in FIG. 7, the ultrasonic receiver 720 includes a receiving end 721, with the receiving end 721 oriented toward the outer side of the garment, or away from a site at which the ultrasonic receiver 720 directly or indirectly contacts the user's limb. In some embodiments, where the garment is a multi-layered structure, the ultrasonic transmitter and the ultrasonic receiver may be located between two layers of the garment, and output directions of the ultrasonic transmitter and the ultrasonic receiver are oriented toward the outer side of the garment. That is, when the user wears the garment, the output end of the ultrasonic transmitter and the receiving end of the ultrasonic receiver are away from a site at which the ultrasonic transmitter and the ultrasonic receiver directly or indirectly contact the user's limb.

As mentioned above, the ultrasonic waves transmitted by the ultrasonic transmitter radiate to the outside world through the output end, and the ultrasonic receiver needs to receive the ultrasonic waves through its receiving end, and since the ultrasonic transmitter and the ultrasonic receiver are located at the user's upper arm lower arm, respectively, if the user straightens his or her arm or leg, the ultrasonic receiver may not be able to receive the ultrasonic waves transmitted by the ultrasonic transmitter. In order to ensure that the ultrasonic waves transmitted by the ultrasonic transmitter can be received by the ultrasonic receiver, and to improve the reliability of the human body posture recognition system, in some embodiments, an angle between a plane on which the output end is located and a plane on which the receiving end is located may be less than 180°. Preferably, the angle between the plane on which the output end is located and the plane on which the receiving end is located may be not greater than 170°. More preferably, the angle between the plane on which the output end is located and the plane on which the receiving end is located may be not greater than 150°. Further preferably, the angle between the plane on which the output end is located and the plane on which the receiving end is located may be not greater than 130°. As shown in FIG. 7, an angle θ illustrated in FIG. 7 may be considered as the angle between the plane on which the output end of the ultrasonic transmitter 710 is located and the plane on which the receiving end of the ultrasonic receiver 720 is located. It should be noted that the plane on which the output end is located is a plane on which a side wall of the housing of the ultrasonic transmitter transmitting the ultrasonic waves is located. For example, the ultrasonic transmitter may include a housing and a vibration unit, and the vibration unit generates the ultrasonic waves based on an electrical signal, and the ultrasonic waves may be radiated externally through a hole portion disposed on the housing, and then a side wall where the hole portion is located may be considered as the output end of the ultrasonic transmitter. For example, the housing may not be provided with the hole portion, and the ultrasonic waves may be radiated externally through a side wall of the housing, and the side wall may be regarded as the output end of the ultrasonic transmitter. Correspondingly, the plane on which the receiving end is located is a plane on which a side wall of the housing of the ultrasonic receiver receiving the ultrasonic waves is located. For example, the ultrasonic receiver may include a housing and a transducer unit, the transducer unit generates an electrical signal based on a sound signal, and the ultrasonic waves transmitted by the ultrasonic transmitter may be transmitted to the housing of the ultrasonic receiver through the hole portion provided on the housing, and act on the transducer unit, and a side wall on which the hole portion is located may be regarded as the receiving end of the ultrasonic receiver. Also, for example, the housing may not be provided with the hole portion, and the ultrasonic waves may be directly received by one side wall of the housing, and the side wall may be regarded as the receiving end of the ultrasonic receiver.

In order for the angle formed between the plane on which the output end is located and the plane on which the receiving end is located to be within a particular angle (e.g., less than 170°), in some embodiments, the ultrasonic transmitter is disposed obliquely relative to a limb contact site on the user below the ultrasonic transmitter and disposed at an inclination toward the ultrasonic receiver, and an angle between a normal direction of the output end and a normal direction of the limb contact site on the user below the ultrasonic transmitter may be not less than 15°. Preferably, the angle between the normal direction of the output end and the normal direction of the limb contact site on the user below the ultrasonic transmitter may be not less than 10°. Further preferably, the angle between the normal direction of the output end and the normal direction of the limb contact site on the user below the ultrasonic transmitter may not be less than 5°. The limb contact site on the user may be the user's skin or garment. For example, when the user wears the garment integrated with the ultrasonic sensor, the limb contact site on the user may be the garment. For example, when the ultrasonic sensor is provided directly with the user's skin, the limb contact site on the user may be the user's skin. As shown in FIG. 7, the ultrasonic transmitter 710 is disposed relative to the limb contact site on the user below the ultrasonic transmitter 710 and disposed obliquely toward the ultrasonic receiver 720, a normal of the output end is p, a normal of the limb contact site on the user below the ultrasonic transmitter is q, and an angle β1 illustrated in FIG. 7 may be regarded as an angle between the normal p and the normal q.

In some embodiments, the ultrasonic receiver is disposed obliquely relative to a limb contact site on the user below the ultrasonic receiver and disposed at an inclination toward the ultrasonic transmitter, and an angle between a normal direction of the receiving end and a normal direction of the limb contact site on the user below the ultrasonic receiver may be not less than 15°. Preferably, the angle between the normal direction of the receiving end and the normal direction of the limb contact site on the user below the ultrasonic receiver may be not less than 10°. Further preferably, the angle between the normal direction of the receiving end and the normal direction of the limb contact site on the user below the ultrasonic receiver may be not less than 5°. As shown in FIG. 7, the ultrasonic receiver 720 is relative to the limb contact site on the user below the ultrasonic receiver 720 and disposed obliquely toward the ultrasonic transmitter 710, with a normal of the receiving end being s and a normal of the limb contact site on the user below the ultrasonic receiver being r, and an angle β2 illustrated in FIG. 7 may be considered as an angle between the normal s and the normal r.

By setting β1 and β2 in the manner described above, the angle between the plane on which the output end is located and the plane on which the receiving end is located is not greater than 170°, and such setting can ensure the ultrasonic receiver may always be able to receive the ultrasonic waves transmitted from the ultrasonic transmitter, which improves the reliability of the human body posture recognition system.

The ultrasonic transmitter 710 and the ultrasonic receiver 720 illustrated in FIG. 7 are rectangular, and the ultrasonic transmitter and the ultrasonic receiver may have other structures and/or shapes.

As shown in FIG. 8, an ultrasonic transmitter 810 and an ultrasonic receiver 820 may be a right-angled trapezoidal body, the right-angled trapezoidal body may be understood to be a structural body in which a rectangular structure is shaved of a prong along a length direction, a width direction, or a thickness direction, and a surface formed by the prong is a trapezoidal surface of the right-angled trapezoidal body. In some embodiments, the trapezoidal surface of the right-angled trapezoidal body (812 and 822 illustrated in FIG. 8) contacts the user's limb. The ultrasonic transmitter 810 is disposed obliquely relative to a limb contact site on the user below the ultrasonic transmitter 810 and disposed obliquely toward the ultrasonic receiver 820, with a normal of an output end 811 being p and a normal of the limb contact site on the user below the ultrasonic transmitter 810 being q, and an angle β1 illustrated in FIG. 8 may be considered as an angle between the normal p and the normal q. The ultrasonic receiver 820 is disposed obliquely relative to a limb contact site on the user below the ultrasonic receiver 820 and disposed obliquely toward the ultrasonic transmitter 810, with a normal of a receiving end 821 being s and a normal of the limb contact site on the user below the ultrasonic receiver 820 being r, and an β2 illustrated in FIG. 8 may be considered as an angle between the normal s and the normal r.

It will be appreciated that the ultrasonic transmitter and the ultrasonic receiver may also be other structures and/or shapes other than those shown in FIG. 7 and FIG. 8 that may satisfy an angle between the normal direction of the output end and the normal direction of the limb contact area on the user below the ultrasonic transmitter, and an angle between the normal direction of the receiving end and the normal direction of the limb contact site on the user below the ultrasonic receiver. For example, a triangular prism, a hexagonal prism, a cylindrical body, or the like.

The ultrasonic transmitter and the ultrasonic receiver shown in FIG. 7 and FIG. 8 may be secured to a human body or a garment by means of, for example, a glue or an adhesive member. In some embodiments, the ultrasonic transmitter and the ultrasonic receiver may also be secured by providing a securing member such that the angle between the normal direction of the output end and the normal direction of the limb contact area on the user below the ultrasonic transmitter is not less than a specific angle (e.g., 5°), and the angle between the normal direction of the receiving end and the normal direction of the limb contact area on the user below the ultrasonic receiver is not less than a specific angle (e.g., 5°). In some embodiments, the securing member may be integrally molded with the ultrasonic sensor (e.g., the ultrasonic transmitter or the ultrasonic receiver) or a structure independently with respect to the ultrasonic sensor. Using FIG. 7 as exemplary, the securing member may be a wedge-shaped structure, the wedge-shaped structure being interposed between the ultrasonic transmitter and the garment in contact with the skin of the user and between the ultrasonic receiver and the garment in contact with the skin of the user.

The ultrasonic sensor not only obtains a bending angle between the two opposite limbs corresponding to the elbow joint or the knee joint and a rotation angle between the two opposite limbs corresponding to the elbow joint or the knee joint, but also may be used to recognize a posture of the upper arm relative to a torso site.

In some embodiments, the ultrasonic transmitter is located in the garment at a location corresponding to an upper arm of a human body, the ultrasonic receiver is located in the garment at a location corresponding to a torso site of the human body, and the ultrasonic transmitter and the ultrasonic receiver cooperate to recognize the posture of the upper arm relative to the torso site. In some embodiments, the ultrasonic receiver may be located at a location in the garment corresponding to the upper arm of the human body, and the ultrasonic transmitter may be located at a location in the garment corresponding to the torso site of the human body. FIG. 9 is a schematic diagram illustrating ultrasonic transmitters and ultrasonic receivers distributed on upper arms and torso sites according to embodiments of the present disclosure. As shown in FIG. 9, an ultrasonic transmitter 910 and an ultrasonic transmitter 930 are located at locations in a garment corresponding to upper arms of the human body, and an ultrasonic receiver 920 and an ultrasonic receiver 940 are located at locations in the garment corresponding to torso sites of the human body.

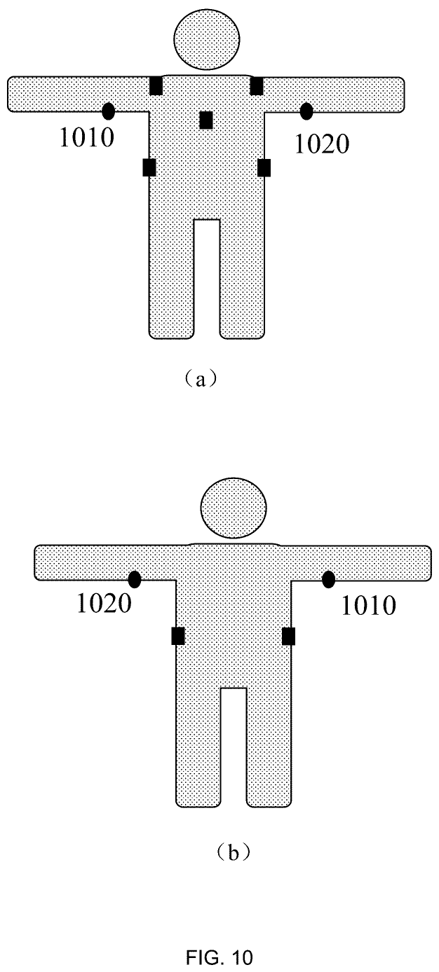

In some embodiments, the ultrasonic receiver may be located at a plurality of locations in the garment corresponding to torso sites of the human body. To achieve more accurate measurements, the ultrasonic receiver may be located at locations shown in FIG. 10.

It is understood that body parts corresponding to a shoulder joint (e.g., the upper arm and torso site) have a more complex form of movement than two opposite limbs corresponding to an elbow joint or a knee joint, e.g., the upper arm may move up and down, forward and backward, rotate, and swing relative to the torso site. The ultrasonic sensor has a limited range of action, so for one ultrasonic transmitter, there is often a measurement dead space if only one ultrasonic receiver is provided, and ultrasonic waves transmitted by the ultrasonic transmitter may not be received in certain states to be accurately localized, and to improve the accuracy of the ultrasonic sensor in recognizing a motion state of the body parts corresponding to the shoulder joint (e.g., the upper arm and the torso site), in some embodiments, ultrasonic sensors correspondingly disposed at the shoulder joint may be a plurality of groups. In some implementations, a group of ultrasonic sensors may include at least one ultrasonic transmitter and a plurality of ultrasonic receivers.

FIG. 10 is a schematic diagram illustrating placement locations of ultrasonic sensors according to some embodiments of the present disclosure, whereas FIG. (a) in FIG. 10 is a schematic diagram illustrating placement locations of ultrasonic sensors on a front side of a human body, and FIG. (b) in FIG. 10 is a schematic diagram illustrating placement locations of ultrasonic sensors on a back side of a human body.

As shown in FIG. 10(a) and FIG. 10(b), an ultrasonic transmitter 1010 and an ultrasonic transmitter 1020 may be located at two upper arms of the human body, respectively, e.g., an ultrasonic receiver (a square black area illustrated in FIG. (a)) may be located at any one or any plurality of locations on the human body such as the anterior side of the left shoulder, the anterior side of the right shoulder, the left lumbar side, the right lumbar side, the chest, or the like. Further, ultrasonic receivers located on the anterior side of the left shoulder and on the left lumbar side may receive ultrasonic waves from an ultrasonic transmitter on a left upper arm (i.e., the ultrasonic transmitter 1010 shown in FIG. 10), ultrasonic receivers located on the anterior side of the right shoulder and the right lumbar side may receive ultrasonic waves from an ultrasonic transmitter of a right upper arm (i.e., the ultrasonic transmitter 1020 shown in FIG. 10), and an ultrasonic receiver located on the chest may both receive the ultrasonic waves from the ultrasonic transmitter on the left upper arm as well as the ultrasonic waves from the ultrasonic transmitter on the right upper arm. When the ultrasonic sensor is integrated into a garment, the ultrasonic transmitter 1010 and the ultrasonic transmitter 1020 may be located at locations in the garment corresponding to the two upper arms of the human body, and the ultrasonic receiver may be located at at least one location in the garment corresponding to at least one of the front side of the left shoulder, the right shoulder front side, left lumbar side, right lumbar side, or the chest. Set up in this way, it can be ensured that at least one of the ultrasonic receivers may receive the ultrasonic waves from the ultrasonic transmitter when the user's arm is making any motion with the shoulder joint as a center, so as to carry out human posture recognition. In some embodiments, in order to further improve the accuracy of a human body posture recognition system in recognizing body motions, one or more ultrasonic transmitters may be provided at each of the user's upper arms, and one or more ultrasonic receivers may be provided at each of the user's left shoulder anterior side, right shoulder anterior side, left lumbar side, right lumbar side, or chest. Preferably, at least two ultrasonic transmitters may be disposed at each of the user's upper arms, and at least three ultrasonic receivers may be disposed at any one of the user's left shoulder anterior side, right shoulder anterior side, left lumbar side, right lumbar side, or chess, the plurality of ultrasonic receivers not being in a same straight line. For example, a count of ultrasonic receivers may be three, with the three ultrasonic receivers distributed in a triangular shape. As another example, a count of ultrasonic receivers may be four, with the four ultrasonic receivers distributed in a triangular or quadrilateral shape. A distance between ultrasonic sensors needs to be limited in order to ensure the measurement accuracy, taking into account a limited dimension of the human body and an angle of the ultrasonic sensors. In some embodiments, a spacing between at least two ultrasonic transmitters two-by-two is not less than 0.5 cm, and a spacing between at least three ultrasonic receivers two-by-two is not less than 0.5 cm, and preferably, a spacing between the at least two ultrasonic transmitters two-by-two is no less than 0.8 cm and the spacing between the at least three ultrasonic receivers two-by-two is no less than 0.8 cm; further preferably, the spacing between the at least two ultrasonic transmitters two-by-two is no less than 1 cm and the spacing between the at least three ultrasonic receivers two-by-two is no less than 1 cm.

In some embodiments, the posture of the upper arm relative to the torso site may include an angle of the upper arm relative to the torso site. The angle of the upper arm relative to the torso site may be understood as an angle formed between an extension direction of the upper arm and a side of the torso site. The determination of the angle of the upper arm relative to the torso site is described herein in conjunction with FIG. 11 for clarity. FIG. 11 is a flowchart illustrating an exemplary process for determining an angle of an upper arm relative to a torso site according to some embodiments of the present disclosure. In some embodiments, a process 1100 may be executed by the processor 120. As shown in FIG. 11, the process 1100 may include following steps.

Step 1110, obtaining a distance between an ultrasonic transmitter and an ultrasonic receiver based on information of ultrasonic waves transmitted by the ultrasonic transmitter, and information of the ultrasonic waves received by the ultrasonic receiver.

More information about obtaining the distance between the ultrasonic transmitter and the ultrasonic receiver based on the information of the ultrasonic waves transmitted by the ultrasonic transmitter and the information of the ultrasonic waves received by the ultrasonic receiver can be referred to FIG. 3 and the related descriptions thereof.

Step 1120, determining the angle of the upper arm relative to the torso site based on the distance between the ultrasonic transmitter and the ultrasonic receiver and location information of the ultrasonic transmitter and the ultrasonic receiver.

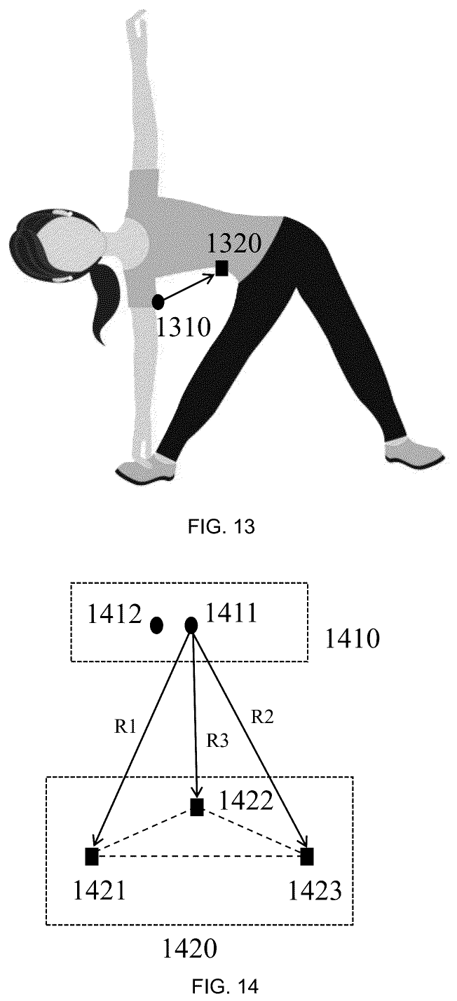

The angle of the upper arm relative to the torso site correlates with a placement location of an ultrasonic sensor. When the ultrasonic transmitter is located on the upper arm and the ultrasonic receiver is located on a side of the torso site (e.g., lumbar side, ribcage, etc.), the angle of the upper arm relative to the torso site refers to an angle of the upper arm relative to the side of the torso site, as shown in FIG. 13, and when the ultrasonic transmitter 1310 is located on the upper arm and the ultrasonic receiver 1320 is located on the lumbar side, the angle of the upper arm with respect to the torso site refers to an angle of the upper arm relative to the lumbar side. When the ultrasonic transmitter is located at the upper arm and the ultrasonic receiver is located at the forehead or the back, the angle of the upper arm relative to the torso site refers to an angle of the arm swinging forward and backward.

Based on the distance between the ultrasonic transmitter and the ultrasonic receiver and the location information of the ultrasonic transmitter and the ultrasonic receiver, the angle of the upper arm relative to the torso site is determined, which can be determined by referring to the relevant contents in FIG. 3.

FIG. 12 is a flowchart illustrating an exemplary process for recognizing a motion state of an upper arm relative to a torso site according to some embodiments of the present disclosure. The motion state of the upper arm relative to the torso site is more complex, and in order to improve the accuracy of a human body posture recognition system in recognizing the motion state, in some embodiments, each group of ultrasonic sensors may include at least two ultrasonic transmitters and at least three ultrasonic receivers. In some embodiments, a process 1200 may be executed by the processor 120. As shown in FIG. 12, the process 1200 may include following steps.

Step 1210, determining location change information of at least two ultrasonic transmitters based on location information of the at least three ultrasonic receivers.

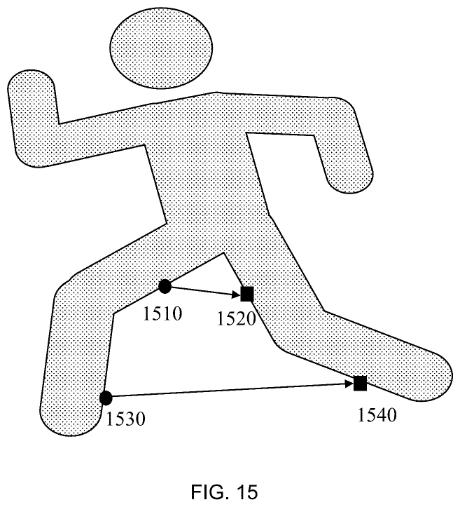

In some embodiments, the at least three ultrasonic receivers are not in the same straight line. As shown in FIG. 14, a line connecting locations where an ultrasonic receiver 1421, an ultrasonic receiver 1422, and an ultrasonic receiver 1423 are located may form a triangle, i.e., the three ultrasonic receivers are not on the same straight line.

A distance between ultrasonic sensors needs to be limited in order to ensure measurement accuracy, taking into account a limited dimension of a human body and an angle of the ultrasonic sensors. In some embodiments, a spacing between the at least two ultrasonic transmitters two-by-two is not less than 0.5 cm, and a spacing between the at least three ultrasonic receivers two-by-two is not less than 0.5 cm, and preferably, the spacing between the at least two ultrasonic transmitters two-by-two is no less than 0.8 cm and the spacing between the at least three ultrasonic receivers two-by-two is no less than 0.8 cm; further preferably, the spacing between the at least two ultrasonic transmitters tow-by-two is no less than 1 cm and the spacing between the at least three ultrasonic receivers two-by-two is no less than 1 cm.

In some embodiments, determining the location change information of the at least two ultrasonic transmitters based on the location information of the at least three ultrasonic receivers may include determining location information of an ultrasonic transmitter based on the location information of the at least three ultrasonic receivers via a formula (3):

$$\{ \begin{matrix}{( x - {x}_{1} )}^{2} + {( y - {y}_{1} )}^{2} + {( z - {z}_{1} )}^{2} = {R}_{1}^{2} \\ {( x - {x}_{2} )}^{2} + {( y - {y}_{2} )}^{2} + {( z - {z}_{2} )}^{2} = {R}_{2}^{2} \\ {( x - {x}_{3} )}^{2} + {( y - {y}_{3} )}^{2} + {( z - {z}_{3} )}^{2} = {R}_{3}^{2}\end{matrix} . \qquad ( 3 )$$

Where, coordinates of the three ultrasonic receivers are (x₁, y₁, z₁), (x₂, y₂, z₂), (x₃, y₃, z₃) are known, coordinates of the ultrasonic transmitter are (x, y, z), and R₁, R₂, and R₃ denote distances between the three ultrasonic receivers and the ultrasonic transmitter.

As shown in FIG. 14, an ultrasonic transmitter 1410 includes an ultrasonic transmitter 1411 and an ultrasonic transmitter 1412, and the ultrasonic receiver 1420 includes the ultrasonic receiver 1421, the ultrasonic receiver 1422, and the ultrasonic receiver 1423. A processor may determine coordinates of the ultrasonic transmitter 1411 and the ultrasonic transmitter 1412, respectively, based on location information of the three ultrasonic receivers via the formula (3).

In some embodiments, the location information of the ultrasonic transmitter may be location information of a corresponding ultrasonic transmitter after the user's upper arm moves. The coordinates of the ultrasonic transmitter 1411 and the ultrasonic transmitter 1412 may be determined in a manner described above when the user's upper arm is in a certain posture.

In some embodiments, determining the location change information of the at least two ultrasonic transmitters based on the location information of the at least three ultrasonic receivers may include determining location information of a reference point based on the location information of the at least three ultrasonic receivers, and determining the location change information of the at least two ultrasonic transmitters based on the location information of the reference point and the location information of the corresponding ultrasonic transmitter after the user's upper arm moves. The reference point refers to any point in a graph formed by the at least three ultrasonic receivers. For example, the reference point may be a geometric center in the graph formed by the at least three ultrasonic receivers. As shown in FIG. 14, the processor may determine, based on coordinate information of the ultrasonic receiver 1421, the ultrasonic receiver 1422, and the ultrasonic receiver 1423, coordinates of a geometric center in the triangle formed by the three ultrasonic receivers, and use the geometric center in the triangle as the reference point. The location change information is a difference in distance from the reference point to different ultrasonic transmitters when the user is in a certain motion posture. For example, the ultrasonic transmitter includes the ultrasonic transmitter 1411 and the ultrasonic transmitter 1412 shown in FIG. 14, and when the user's upper arm is in an arm-expanding chest expansion motion, the location change information of the two ultrasonic transmitters includes a difference between a distance between the reference point and the ultrasonic transmitter 1411 and a difference between the reference point and the ultrasonic transmitter 1412. As an exemplary illustration only, when the user stands naturally with an arm naturally straightened downward, distances between the reference point and the two ultrasonic transmitters, respectively (e.g., X1′ and X2′) may be regarded as approximately equal, and when the user's upper arm is rotated with a shoulder joint as the center, the two ultrasonic transmitters rotate with respect to the reference point, and at this time, the two distances X1′ and X2′ between the ultrasonic transmitters and the reference point change, and corresponding values of X1′ and X2′ are not equal, then based on a magnitude and positive and negative of a difference between X1′ and X2′ (i.e., the location change information of the two ultrasonic transmitters), a degree of rotation and a motion direction of the upper arm relative to the torso site may be determined. Specifics regarding the rotation angle of the upper arm with respect to the torso site can be found in FIG. 5 to FIG. 6C.

Step 1220, recognizing a motion state of the upper arm relative to the torso site based on the location change information of the at least two ultrasonic transmitters.

The motion state of the upper arm relative to the torso site may include the rotation angle of the upper arm relative to the torso site, the motion direction, or the like. The rotation angle may be used to characterize a degree to which a specific location of the upper arm (e.g., a region of the upper arm corresponding to the axilla) is rotated relative to the torso site centered on the shoulder joint.

In some embodiments, the processor may determine a distance (e.g., X1′ and X2′) between the reference point and the two ultrasonic transmitters, respectively, based on a location (coordinates) of the reference point and the location information of the two ultrasonic transmitters, and determine the rotation angle of the upper arm relative to the torso site via the formula (2). For example, the processor may take a distance between the ultrasonic transmitter 1411 and the reference point as X1′ and a distance between the ultrasonic transmitter 1412 and the reference point as X2′, and then substitute X1=X1′ and X2=X2′ into the formula (2) to determine the rotation angle of the upper arm relative to the torso site.

It is also important to note that the result determined by the formula (2) is a relative quantity that may indicate the degree of rotation of the upper arm, and is not a precise rotation angle of the upper arm relative to the torso site.

In some embodiments, the processor may determine, based on the rotation angle of the upper arm relative to the torso site, location information of the ultrasonic transmitter and the ultrasonic receiver, information of ultrasonic waves transmitted by the ultrasonic transmitter, and information of the ultrasonic waves received by the ultrasonic receiver, a motion state of the upper arm relative to the torso site. For example, when an ultrasonic receiver located at the chest receives ultrasonic waves transmitted by an ultrasonic transmitter located at the upper arm, the processor may determine that the upper arm extends forward relative to the torso site. Further, after the ultrasonic waves transmitted by the ultrasonic transmitter are received by the ultrasonic receiver in the chest, based on the information of the ultrasonic waves transmitted by the ultrasonic transmitter and the information of the ultrasonic waves received by the ultrasonic receiver, a distance between the ultrasonic transmitter and the ultrasonic receiver in the chest may be determined, thereby determining an angle of forward extension of the arm. Further, the processor may determine, based on the rotation angle of the upper arm relative to the torso site, the rotation angle of the upper arm (e.g., a corresponding part of the armpit of the upper arm) relative to the torso site (the lumbar side part) may be determined, which may, in turn, determines the motion state of the upper arm relative to the torso site.

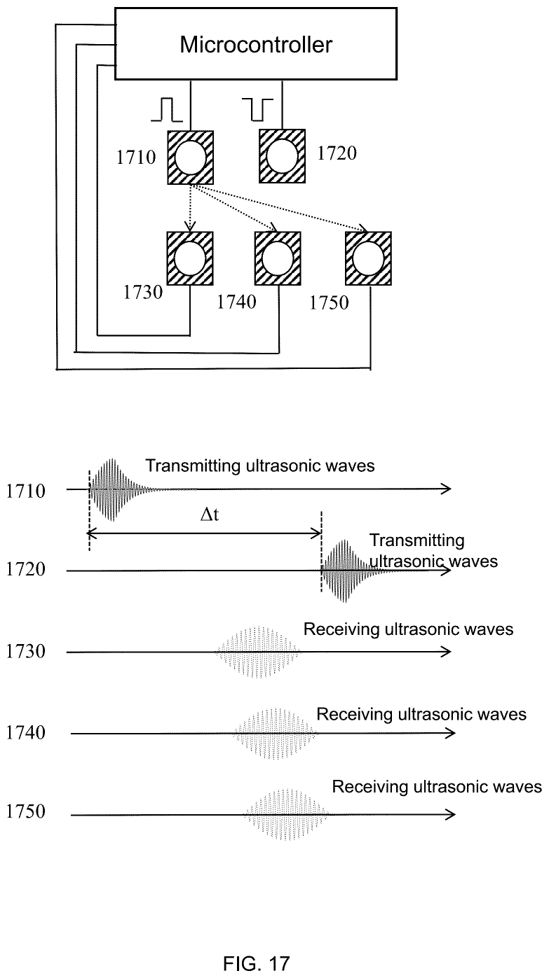

In some embodiments, in order to avoid crosstalk of signals between different ultrasonic transmitters, different ultrasonic transmitters may be time-shared and multiplexed so that there is a certain time interval between signals transmitted by different ultrasonic transmitters. In this case, if there are too many ultrasonic transmitters, this will result in a decrease in a sampling rate, i.e., the greater the count of ultrasonic transmitters, the fewer signals the ultrasonic receiver collects per second from the same ultrasonic transmitter. Therefore, in order to increase the sampling rate of the same ultrasonic transmitter, the fewer ultrasonic transmitters used in an overall layout, the better, and by arranging the ultrasonic transmitter on the upper arm, the count of ultrasonic transmitters can be saved to the greatest content while ensuring that a plurality of postures can be recognized. More information of the time-sharing multiplexing of ultrasonic transmitters can be found in FIG. 17 and the related descriptions thereof.

At the same time, the above design can make a calculation result more accurate. When using the formula (2), its localization accuracy requires that R₁, R₂, and R₃ be distances between the ultrasonic transmitter and a plurality of ultrasonic receivers at a same moment, i.e., R₁, R₂, and R₃ are determined at the same moment if possible. The larger the time gap, the larger the error. Using one ultrasonic transmitter to transmit ultrasonic waves and three ultrasonic receivers to receive the ultrasonic waves can maximize the simultaneity of the ultrasonic waves; on the contrary, if using three ultrasonic transmitters to transmit ultrasonic waves and one ultrasonic receiver to receive the ultrasonic waves, under the influence of time-sharing multiplexing of the ultrasonic transmitter, there is a large time difference between a transmission time of the 3 ultrasonic transmitters, so obtained R₁, R₂, R₃ is not data the same moment, and a calculation error may be larger.

By using the ultrasonic sensor and designing its placement location, it may also be used to determine a posture of the user's leg.

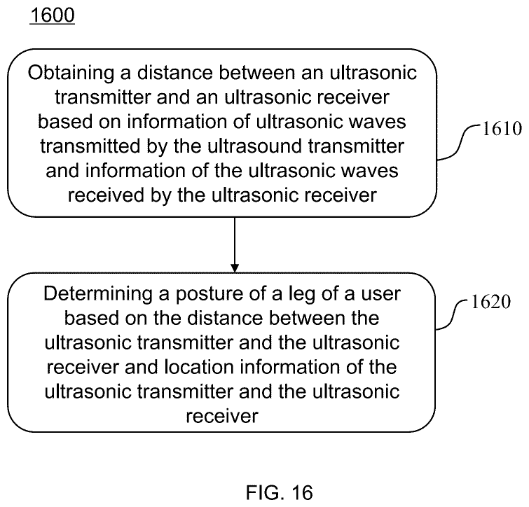

FIG. 16 is a flowchart illustrating an exemplary process for determining a posture of a user's leg according to some embodiments of the present disclosure. In some embodiments, a process 1600 may be executed by the processor 120. As shown in FIG. 16, the process 1600 may include following steps.

Step 1610, obtaining a distance between an ultrasonic transmitter and an ultrasonic receiver based on information of ultrasonic waves transmitted by the ultrasonic transmitter and information of the ultrasonic waves received by the ultrasonic receiver.

In some embodiments, the ultrasonic transmitter and ultrasonic receiver may be located at user's two thighs or two calves, respectively, in order to recognize a posture of the user's legs. For example, one of the ultrasonic transmitter and the ultrasonic receiver is located at a left thigh and the other is located at a right thigh. As another example, one of the ultrasonic transmitter and the ultrasonic receiver is located at a left calf and the other is located at a right calf.

In some embodiments, when an ultrasonic sensor is integrated into a garment, the ultrasonic transmitter and the ultrasonic receiver may be located at two trouser legs in the garment to recognize the posture of the user's legs. For example, one of the ultrasonic transmitter and the ultrasonic receiver is located at a left trouser leg corresponding to the left thigh, and the other is located at a right trouser leg corresponding to the right thigh. As another example, one of the ultrasonic transmitter and the ultrasonic receiver is located at a left trouser leg corresponding to the left calf and the other is located at a right trouser leg corresponding to the right calf. As shown in FIG. 15, an ultrasonic transmitter 1510 and an ultrasonic receiver 1520 may be located at trouser legs in the garment corresponding to the thighs, and an ultrasonic transmitter 1530 and an ultrasonic receiver 1540 may be located at trouser legs in the garment corresponding to the calves.

More information about obtaining the distance between the ultrasonic transmitter and the ultrasonic receiver based on the information of the ultrasonic waves transmitted by the ultrasonic transmitter, and the information of the ultrasonic waves received by the ultrasonic receiver can be referred to the relevant contents in FIG. 3.

Step 1620, determining a posture of a leg of the user based on the distance between the ultrasonic transmitter and the ultrasonic receiver and location information of the ultrasonic transmitter and the ultrasonic receiver.

The posture of the leg refers to information related to a location and a posture of both legs. For example, angles formed between extension directions of the legs, a motion state of the legs (e.g., standing, walking, running, etc.), and a posture of the legs (crossed legs, parallel legs, etc.).

In some embodiments, the processor may determine the posture of the user's leg based on the location information of the ultrasonic transmitter and the ultrasonic receiver, and by comparing the distance between the ultrasonic transmitter and the ultrasonic receiver to a threshold value. For example, one group of ultrasonic sensors (e.g., a first ultrasonic transmitter and a first ultrasonic receiver) is provided at the user's left thigh and right thigh, and another group of ultrasonic sensors (e.g., a second ultrasonic transmitter and a second ultrasonic receiver) is provided at the user's left calf and right calf. The processor may determine that the posture of the user's legs is parallel based on a distance between the first ultrasonic transmitter and the first ultrasonic receiver being less than a threshold (e.g., 4 cm), and a distance between the second ultrasonic transmitter and the second ultrasonic receiver being less than a threshold (e.g., 6 cm). It should be noted that the threshold value may be set based on different users.

By setting the ultrasonic transmitter and the ultrasonic receiver at different locations of the human body, posture recognition of different parts of the human body can be realized. As described above, by setting the ultrasonic transmitter and the ultrasonic receiver at two opposite limbs corresponding to the user's elbow joint or knee joint, respectively, a motion posture of the user's arm or leg may be recognized; by setting the ultrasonic transmitter at the upper arm of the human body, and the ultrasonic receiver is set at the torso site of the human body, a posture of the upper arm relative to the torso site may be recognized; by setting the ultrasonic transmitter and the ultrasonic receiver at the two thighs or the two calves of the user, respectively, the posture of the user's legs may be recognized.

By simultaneously setting the ultrasonic transmitter and the ultrasonic receiver at different parts of the human body as described above, a motion posture of the user's arm or leg, a posture of the upper arm relative to the torso site, and the posture of the user's leg can be simultaneously obtained, and thus recognizing a posture of a whole body of the user.

A plurality of ultrasonic transmitters are required to transmit ultrasonic waves simultaneously in this system, and in some embodiments, in order to avoid mutual crosstalk of ultrasonic waves between different ultrasonic transmitters, time-sharing multiplexing may be used to differentiate between different transmission times of ultrasonic waves in a time domain.

FIG. 17 is a schematic diagram illustrating realizing time-sharing multiplexing based on an ultrasonic sensor according to some embodiments of the present disclosure.

Time-sharing multiplexing refers to different devices (e.g., ultrasonic transmitters) generating ultrasonic waves at different times. In some embodiments, the time-sharing multiplexing may be implemented based on different transmission times of ultrasonic waves transmitted from different ultrasonic transmitters in each group of ultrasonic sensors.

In some embodiments, a plurality of ultrasonic transmitters are included in each group of ultrasonic sensors, and there is a time interval between time points of the plurality of ultrasonic transmitters transmitting ultrasonic waves. As shown in FIG. 17, a microcontroller controls operation states of an ultrasonic transmitter 1710 and an ultrasonic transmitter 1720, respectively, by means of a control signal, and when the ultrasonic transmitter 1710 transmits ultrasonic waves, the ultrasonic transmitter 1720 is in a dormant state and does not transmit ultrasonic waves, then at this time an ultrasonic receiver 1730, an ultrasonic receiver 1740, and an ultrasonic receiver 1750 receive the ultrasonic waves from the ultrasonic transmitter 1710, and after a time Δt, the ultrasonic transmitter 1720 begins to transmit ultrasonic waves.

Being limited by a dimension of a human body, a distance L between the ultrasonic transmitter and the ultrasonic receiver is less than 1 m, and then a transmission time t of the ultrasonic waves is less than 2.9 ms. Therefore, in order to prevent crosstalk between different signals, it is necessary to limit a time interval between ultrasonic waves transmitted by two adjacent ultrasonic transmitters. In some embodiments, the time interval between two adjacent ultrasonic transmitters transmitting ultrasonic waves is greater than 2.9 ms. Set up in this way, even if all ultrasonic receivers are always in an operation state, they may receive and differentiate ultrasonic waves from different ultrasonic transmitters in real-time, and the microcontroller may calculate a change in a relative distance and location with a corresponding ultrasonic transmitter based on the ultrasonic waves from the ultrasonic receivers and a time difference.

In some embodiments, frequencies of the ultrasonic waves transmitted from different ultrasonic transmitters may be set in order to avoid mutual crosstalk of the ultrasonic waves between the different ultrasonic transmitters. For example, each group of ultrasonic sensors includes a plurality of ultrasonic transmitters, and the plurality of ultrasonic transmitters transmit ultrasonic waves at different frequencies. For example, the ultrasonic waves transmitted by the ultrasonic transmitter 1710 has a frequency of 50 kHz to 140 kHz, and the ultrasonic waves transmitted by the ultrasonic transmitter 1720 has a frequency of 200 kHz to 300 kHz. When the ultrasonic receiver receives ultrasonic waves, the processor may locate an ultrasonic transmitter that transmits ultrasonic waves based on a frequency of the ultrasonic waves, and further determine a distance and a location of the ultrasonic transmitter from the ultrasonic receiver.

In some embodiments, a code of ultrasonic waves transmitted from the ultrasonic transmitter may be set in order to prevent ultrasonic waves from different ultrasonic transmitters from cross talking each other. For example, a plurality of ultrasonic transmitters are included in each group of ultrasonic sensors, and ultrasonic waves transmitted by the plurality of ultrasonic transmitters have different codes. For example, the processor may encode the ultrasonic waves, via the group of universal codec ICs, such that the plurality of ultrasonic transmitters may transmit ultrasonic waves with different codes.

Understandably, a human body posture recognition system may use a plurality of groups of ultrasonic sensors, and crosstalk of signals may occur in a plurality of ultrasonic transmitters included in the plurality of groups of ultrasonic sensors, and in order to avoid such a situation, a plurality of ultrasonic transmitters in different groups may be set.

In some embodiments, a plurality of ultrasonic transmitters are included in each group of ultrasonic sensors, and there is a time interval between time points at which the plurality of ultrasonic transmitters in different groups transmit ultrasonic waves. For example, if crosstalk may occur between signals from a first ultrasonic transmitter in a group A and a second ultrasonic transmitter in a group B, time points at which ultrasonic waves transmitted by the first ultrasonic transmitter in the group A and by the second ultrasonic transmitter in the group B may be set with a preset time interval.

In some embodiments, each group of ultrasonic sensors includes a plurality of ultrasonic transmitters, and the plurality of ultrasonic transmitters in different groups transmit ultrasonic waves at different frequencies. For example, the plurality of ultrasonic transmitters in the group A transmits ultrasonic waves at a frequency of 50 kHz to 140 kHz, and the plurality of ultrasonic transmitters in the group B transmits ultrasonic waves at a frequency of 200 kHz to 300 KHz.

In some embodiments, a plurality of ultrasonic transmitters are included in each group of ultrasonic sensors, and ultrasonic waves transmitted from the plurality of ultrasonic transmitters in different groups have different codes. For example, the plurality of ultrasonic transmitters in the group A transmits codes I, II, and III, and the plurality of ultrasonic transmitters in the group B transmits codes V, VI, and VII.

With the time-sharing multiplexing described above, as well as the setting of the frequency and code of the ultrasonic waves transmitted by the ultrasonic transmitter, it is possible to avoid crosstalk of the signals and to prevent incorrect calculations of the distance between the ultrasonic transmitter and the ultrasonic receiver from affecting the recognition of the user's posture.

The basic concepts have been described above, and it is apparent to those skilled in the art that the foregoing detailed disclosure serves only as an example and does not constitute a limitation of the present disclosure. Although not explicitly stated here, those skilled in the art may make various modifications, improvements and amendments to the present disclosure. These alterations, improvements, and modifications are intended to be suggested by the present disclosure, and are within the spirit and scope of the exemplary embodiments of the present disclosure.

Moreover, certain terminology has been used to describe embodiments of the present disclosure. As in “an embodiment”, “one embodiment”, and/or “some embodiments” means a feature, structure, or characteristic associated with at least one embodiment of the present disclosure. Accordingly, it should be emphasized and noted that two or more references to “an embodiment” or “one embodiment” or “an alternative embodiment” in different places in the present disclosure do not necessarily refer to the same embodiment. In addition, some features, structures, or features in the present disclosure of one or more embodiments may be appropriately combined.

Furthermore, it will be appreciated by those skilled in the art that aspects of the present disclosure may be illustrated and described by a number of patentable categories or circumstances, including any new and useful process, machine, product, or combination of substances, or any of their new and useful improvements. Accordingly, all aspects of the present disclosure may be performed entirely by hardware, may be performed entirely by softwares (including firmware, resident softwares, microcode, etc.), or may be performed by a combination of hardware and softwares. The above hardware or softwares can be referred to as “data block”, “module”, “engine”, “unit”, “component” or “system”. In addition, aspects of the present disclosure may appear as a computer product located in one or more computer-readable media, the product including computer-readable program code.

Computer storage media may comprise a propagated data signal with a computer program encoded within it, e.g., on a baseband or as part of a carrier. The propagation signal may have a variety of manifestations, including an electromagnetic form, an optical form, and the like, or suitable combinations thereof. The computer storage medium may be any computer-readable medium, other than a computer-readable storage medium, which may be used by connecting to an instruction-executing system, device, or apparatus for communicating, propagating, or transmitting for use. Program code disposed on the computer storage medium may be disseminated via any suitable medium, including radio, cable, fiber optic cable, RF, or the like, or a combination of any of the foregoing.

The computer program code required for the operation of the various portions of the present disclosure may be written in any one or more of a number of programming languages, including object-oriented programming languages such as Java, Scala, Smalltalk, Eiffel, JADE, Emerald, C++, C#, VB.NET, Python, etc., conventional procedural programming languages such as C, Visual Basic, Fortran 2003, Perl, COBOL 2002, PHP, ABAP, dynamic programming languages such as Python, Ruby and Groovy, or other programming languages. The program code can be run entirely on the user's computer, or as a stand-alone package on the user's computer, or partially on the user's computer and partially on a remote computer, or entirely on a remote computer or server. In the latter case, the remote computer can be connected to the user's computer through any form of network, such as a local area network (LAN) or wide area network (WAN), or connected to an external computer (e.g., via the Internet), or in a cloud computing environment, or used as a service such as software as a service (SaaS).

In addition, the order of processing elements and sequences, the use of numerical letters, or the use of other names described herein are not intended to qualify the order of the processes and methods of the present disclosure unless expressly stated in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

Some embodiments use numbers to describe the number of components, attributes, and it should be understood that such numbers used in the description of the embodiments are modified in some examples by the modifiers “about”, “approximately”, or “substantially”. Unless otherwise noted, the terms “about,” “approximately,” or “substantially” indicates that a ±20% variation in the stated number is allowed. Correspondingly, in some embodiments, the numerical parameters used in the present disclosure and claims are approximations, which approximations are subject to change depending on the desired characteristics of individual embodiments. In some embodiments, the numerical parameters should take into account the specified number of valid digits and employ general place-keeping. While the numerical domains and parameters used to confirm the breadth of their ranges in some embodiments of the present disclosure are approximations, in specific embodiments, such values are set to be as precise as possible within a feasible range.

For each patent, patent application, patent application disclosure, and other material cited in the present disclosure, such as articles, books, specifications, publications, documents, and the like, the entire contents are hereby incorporated herein by reference. Except for application history documents that are inconsistent with or conflict with the contents of the present disclosure, and except for documents (currently or hereafter appended to the present disclosure) that limit the broadest scope of the claims of the present disclosure. It should be noted that in the event of any inconsistency or conflict between the descriptions, definitions, and/or use of terms in the materials appurtenant to this application and those set forth herein, the descriptions, definitions, and/or use of terms in the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A human body posture recognition system, comprising:
at least one group of ultrasonic sensors, each group of ultrasonic sensors including:
an ultrasonic transmitter configured to transmit ultrasonic waves and an ultrasonic receiver configured to receive the ultrasonic waves, wherein the ultrasonic transmitter and the ultrasonic receiver are located at different parts of a user's body; and
a processor configured to recognize a posture of the user based on location information of the ultrasonic transmitter and the ultrasonic receiver, information of the ultrasonic waves transmitted by the ultrasonic transmitter, and information of the ultrasonic waves received by the ultrasonic receiver.

2. The system of claim 1, further comprising a garment, wherein the at least one group of ultrasonic sensors is integrated into the garment.

3. The system of claim 2, wherein the ultrasonic transmitter and the ultrasonic receiver are disposed at a sleeve or a trouser leg of the garment, and when the user wears the garment, the ultrasonic transmitter and the ultrasonic receiver are located at two opposite limbs corresponding to an elbow joint or a knee joint of the user, respectively.

4. The system of claim 3, wherein the recognizing the posture of the user based on the location information of the ultrasonic transmitter and the ultrasonic receiver, the information of the ultrasonic waves transmitted by the ultrasonic transmitter, and the information of the ultrasonic waves received by the ultrasonic receiver includes:
obtaining a distance between the ultrasonic transmitter and the ultrasonic receiver based on the information of the ultrasonic waves transmitted by the ultrasonic transmitter and the information of the ultrasonic waves received by the ultrasonic receiver; and
obtaining a bending angle between the two opposite limbs corresponding to the elbow joint or the knee joint based on the distance between the ultrasonic transmitter and the ultrasonic receiver, and the location information of the ultrasonic transmitter and the ultrasonic receiver.

5. The system of claim 3, wherein each group of ultrasonic sensors includes an ultrasonic transmitter and at least two ultrasonic receivers, the at least two ultrasonic receivers including a first ultrasonic receiver and a second ultrasonic receiver, and the recognizing the posture of the user based on the location information of the ultrasonic transmitter and the ultrasonic receiver, the information of the ultrasonic waves transmitted by the ultrasonic transmitter, and the information of the ultrasonic waves received by the ultrasonic receiver includes:
determining a first distance between the ultrasonic transmitter and the first ultrasonic receiver and a second distance between the ultrasonic transmitter and the second ultrasonic receiver based on the information of the ultrasonic waves transmitted by the ultrasonic transmitter, information of ultrasonic waves received by the first ultrasonic receiver, and information of ultrasonic waves received by the second ultrasonic receiver; and
determining a rotation angle between the two opposite limbs corresponding to the elbow joint or the knee joint based on the first distance and the second distance.

6. The system of claim 3, wherein inner sides of the two opposite limbs corresponding to the elbow joint or the knee joint refer to two opposite lateral portions of the limb corresponding to the elbow joint or the knee joint when the elbow joint or the knee joint is in a bending state, and the ultrasonic transmitter and the ultrasonic receiver are located at the inner sides of the two opposite limbs corresponding to the elbow joint or the knee joint.

7. The system of claim 3, wherein the distance between the ultrasonic transmitter and the ultrasonic receiver is no less than 10 cm when the user straightens his or her arm or leg.

8. The system of claim 3, wherein
the ultrasonic transmitter includes an output end for transmitting the ultrasonic waves, and the output end is away from the garment;
the ultrasonic receiver includes a receiving end for receiving the ultrasonic waves, and the receiving end is away from the garment; and
an angle between a plane on which the output end is located and a plane on which the receiving end is located is not greater than 170°.

9. The system of claim 8, wherein the ultrasonic transmitter is disposed obliquely relative to a limb contact site on the user below the ultrasonic transmitter and disposed at an inclination toward the ultrasonic receiver, and an angle between a normal direction of the output end and a normal direction of the limb contact site on the user below the ultrasonic transmitter is not less than 5°.

10. The system of claim 8, wherein the ultrasonic receiver is disposed obliquely relative to a limb contact site on the user below the ultrasonic receiver and disposed at an inclination toward the ultrasonic transmitter, and an angle between a normal direction of the receiving end and a normal direction of the limb contact site on the user below the ultrasonic receiver is not less than 5°.

11. The system of claim 2, wherein the ultrasonic transmitter is located in the garment at a location corresponding to an upper arm of a human body, the ultrasonic receiver is located in the garment at a location corresponding to a torso site of the human body, and the ultrasonic transmitter and the ultrasonic receiver cooperate to recognize a posture of the upper arm relative to the torso site.

12. The system of claim 11, wherein the posture of the upper arm relative to the torso site includes an angle of the upper arm relative to the torso site, and the recognizing the posture of the upper arm relative to the torso site includes:
obtaining a distance between the ultrasonic transmitter and the ultrasonic receiver based on the information of the ultrasonic waves transmitted by the ultrasonic transmitter and the information of the ultrasonic waves received by the ultrasonic receiver; and
determining the angle of the upper arm relative to the torso site based on the distance between the ultrasonic transmitter and the ultrasonic receiver and the location information of the ultrasonic transmitter and the ultrasonic receiver.

13. The system of claim 11, wherein the ultrasonic transmitter includes at least two ultrasonic transmitters and the ultrasonic receiver includes at least three ultrasonic receivers, and the recognizing the posture of the upper arm relative to the torso site includes:
determining location change information of the at least two ultrasonic transmitters based on location information of the at least three ultrasonic receivers; and
recognizing a motion state of the upper arm relative to the torso site based on the location change information of the at least two ultrasonic transmitters.

14. The system of claim 12, wherein the at least three ultrasonic receivers are not in a same straight line.

15. The system of claim 13, wherein the at least two ultrasonic transmitters are spaced no less than 1 cm apart from each other, and the at least three ultrasonic receivers are spaced no less than 1 cm apart from each other.

16. The system of claim 11, wherein in the garment, the location corresponding to the torso site of the human body includes at least one of a front side of a left shoulder, a front side of a right shoulder, a left lumbar side, a right lumbar side, or a chest.

17. The system of claim 2, wherein the ultrasonic transmitter and the ultrasonic receiver are located at two trouser legs of the garment, respectively, and the recognizing the posture of the user based on the location information of the ultrasonic transmitter and the ultrasonic receiver, the information of the ultrasonic waves transmitted by the ultrasonic transmitter, and the information of the ultrasonic waves received by the ultrasonic receiver includes:
obtaining a distance between the ultrasonic transmitter and the ultrasonic receiver based on the information of the ultrasonic waves transmitted by the ultrasonic transmitter and the information of the ultrasonic waves received by the ultrasonic receiver; and
determining a posture of a leg of the user based on the distance between the ultrasonic transmitter and the ultrasonic receiver, and the location information of the ultrasonic transmitter and the ultrasonic receiver.

18. The system of claim 1, wherein each group of ultrasonic sensors includes a plurality of ultrasonic transmitters, and the plurality of ultrasonic transmitters transmit ultrasonic waves at time intervals.

19. (canceled)

20. The system of claim 1, wherein each group of ultrasonic sensors includes a plurality of ultrasonic transmitters, and the plurality of ultrasonic transmitters transmit ultrasonic waves at different frequencies.

21. The system of claim 1, wherein each group of ultrasonic sensors includes a plurality of ultrasonic transmitters, and ultrasonic waves transmitted by the plurality of ultrasonic transmitters have different codes.
